SEQUENCE LISTING (4 sequences)

SEQ ID NO: 1 - protein, 21 aa, Artificial
  SYNTHETIC CONSTRUCT
ATCCTGGCAAGCCCTAAGACC

SEQ ID NO: 2 - protein, 21 aa, Artificial
  SYNTHETIC CONSTRUCT
TGGTAGCGTTTGCTGATCCCG

SEQ ID NO: 3 - protein, 24 aa, Artificial
  SYNTHETIC CONSTRUCT
TTGTAACCAACTGGGACGATATGG

SEQ ID NO: 4 - protein, 21 aa, Artificial
  SYNTHETIC CONSTRUCT
CACAATGCCAGTGGTACGACC

(19) United States
(12) Patent Application Publication
(10) Pub. No.: US 2007/0254055 A1
(43) Pub. Date: Nov. 1, 2007

(54) Therapeutic Avenanthramide Compounds

(75) Inventor: Mohsen Meydani, Newton, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: TRUSTEES OF TUFTS COLLEGE, Medford, MA (US)

(21) Appl. No.: 11/687,910

(22) Filed: Mar. 19, 2007

Related U.S. Application Data
(63) Continuation-in-part of application No. 11/257,918, filed on Oct. 25, 2005.
Application No. 11/257,918 is a continuation-in-part of application No. 10/995,722, filed on Nov. 22, 2004.
(60) Provisional application No. 60/625,484, filed on Nov. 5, 2004.
(60) Provisional application No. 60/524,327, filed on Nov. 21, 2003.

(51) Int. Cl. A61K 31/165; A61K 36/00; A61P 1/00

(52) U.S. Cl. 424/776; 514/617; 564/161

(57) ABSTRACT

Methods and compositions are disclosed for inhibiting proliferation of human colon adenocarcinoma cells, reducing pro-inflammatory molecules, adhesion molecules, and vascular smooth muscle cell proliferation, and for increasing NO production. The present invention describes the use of phenolic compositions, purified from oats or synthetically produced, to decrease the effective amount of pro-inflammatory molecules and/or cell adhesion molecules. Alternatively, an alcoholic extract or concentrate from oats can be used. The methods of the present invention can be used as a treatment or prophylaxis of a wide variety of disorders associated with inflammatory states and/or with a lack of or need for nitric oxide (NO), such as inflammatory conditions, pain, free radical associated disorders, cardiovascular diseases, autoimmune disorders, pathological platelet aggregation, pathological vasoconstriction, vascular effects of diabetes, stroke, atherosclerosis, hypertension, abnormal vasospasm, and restenosis after angioplasty.

### RELATED APPLICATIONS

This application is a continuation in part of U.S. application Ser. No. 11/257,918, filed Oct. 25, 2005, which is a continuation in part of U.S. application Ser. No. 10/995,722, filed Nov. 22, 2004, which claims priority from U.S. Provisional Application Ser. No. 60/524,327, filed Nov. 21, 2003, entitled “Oat-Derived Therapeutic Compositions” and U.S. Provisional Application Ser. No. 60/625,484, filed Nov. 5, 2004, entitled “Modulation of Nitric Oxide Production And Cell Proliferation Using Oat-Derived Phenolic Compounds,” each of which are hereby incorporated by reference in their entirety.

### GOVERNMENT SUPPORT

This invention was made with government support under 58-1950-9-001 awarded by the United States Department of Agriculture. The government has certain rights in the invention.

### FIELD OF THE INVENTION

The present invention concerns phenolic compositions and extracts derived from oats and methods of using such compositions as therapeutic agents.

### BACKGROUND OF THE INVENTION

Cancer is a the second leading cause of death in the United States. Every year, over 100,000 new cases of colon cancer, also known as cancer of the large bowel and colorectal cancer, are diagnosed. According to the American Cancer Society, colon cancer is second to lung cancer as a cause of cancer death, with more than 55,000 Americans dying from colon cancer in 2006. The chances of developing colorectal cancer increases with age. People over the age of 70 have about 5% chance of developing colon cancer.

The treatment of colon cancer once diagnosis is made depends on the extent of the cancer's invasion of the colon tissue, lymph nodes, and metastasis to other organs such as the liver. The survival rate for patients diagnosed with early-stage cancer is about 90% survival after 5 years. The five-year survival rate drops if the cancer is not detected until the cancer has spread beyond the mucosal layer of the colon, and drops significantly further if, when detected, the cancer has spread beyond the colon to the lymph nodes and beyond. Thus, it is critical to diagnose colon cancer early to increase the likelihood of a positive prognosis and outcome.

There are currently three primary treatments available for patients with cancer of the colon. These methods include surgical removal of the cancerous cells, radiation therapy using high-dose x-rays or other high energy radiation to kill the cancer cells, and chemotherapy. These treatments depend upon the stage of the cancer and the health of the individual seeking the treatment. These treatments can be extremely costly and unpredictable.

Accordingly, there exists a need for better methods and compositions for the treatment of cancer. In particular, new compositions capable of inhibiting proliferation of colon cancer cells would satisfy a long-felt therapeutic need.

### SUMMARY OF THE INVENTION

Methods and compositions are disclosed for reducing pro-inflammatory molecules, adhesion molecules, and vascular smooth muscle cell proliferation, and for increasing NO production. The present invention describes the use of phenolic compositions, purified from oats or synthetically produced, to decrease the effective amount of pro-inflammatory molecules and/or cell adhesion molecules. Alternatively, an alcoholic extract or concentrate from oats can be used. The methods of the present invention can be used as a treatment or prophylaxis of a wide variety of disorders associated with inflammatory states and/or with a lack of or need for nitric oxide (NO), such as inflammatory conditions, pain, free radical associated disorders, cardiovascular diseases, autoimmune disorders, pathological platelet aggregation, pathological vasoconstriction, vascular effects of diabetes, stroke, atherosclerosis, hypertension, abnormal vasospasm, and restenosis after angioplasty.

In one aspect of the present invention, human aortic smooth muscle cell (HASMC) proliferation can be reduced and NO production can be increased using an alcoholic oat extract and/or phenolic compounds. In another aspect, pro-inflammatory cytokines and cell adhesion molecules can be inhibited using an alcoholic oat extract and/or phenolic compounds. Non-limiting examples of pro-inflammatory molecules include IL-6, IL-8, and MCP-1. Non-limiting examples of cell adhesion molecules include ICAM-1, VCAM-1, and E-selectin.

The invention makes use of an extract from oats and/or the synthetic purification of these phenolic compounds. The phenolic compounds can have the core structure shown below: 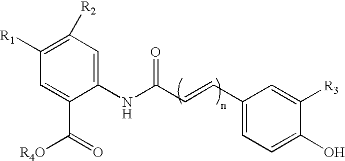
where n is less than or equal to six, and R₁, R₂, and R₃ depict the various side chains which can include, but are not limited to, a hydroxide, an aliphatic group, an aromatic group, an acyl group, an alkoxy group, an alkylene group, an alkenylene group, an alkynylene group, a hydroxycarbonylalkyl group, an anhydride, an amide, an amine, and a heterocyclic aromatic group, and R₄ is a hydrogen or an alkyl group. In some embodiments, n is less than three and the side chains are selected from the group consisting of H, OH, or OCH₃. In some embodiments, the phenolic compound is Avn-C, wherein n is 1, R₁ is OH, R₂ is hydrogen, R₃ is OH, and R₄ is hydrogen. In preferred embodiments, the alkyl ester form of the phenolic compounds can be used. In some embodiments, n is 1, R₁ is OH, R₂ is hydrogen, R₃ is OH, and R₄ is an alkyl group. The alkyl group is preferably a lower alkyl group which can, for example, be selected from the group consisting of methyl, ethyl, propyl, and butyl. Increased potency can be achieved with the alkyl ester form of the phenolic compounds as compared to the non-alkylated form of the phenolic compound.

In a preferred embodiment, the phenolic compounds are avenanthramides. The avenanthramides may be purified from grain. More than 40 distinct avenanthramides have been isolated from oat grains (Collins. J. Agric. Food Chem. 37: 60-66 (1989)). In another aspect of the invention, the avenanthramides are produced synthetically. Methods of synthesis are known in the art as illustrated in U.S. Pat. Nos. 6,096,770 and 6,127,392 as well as Japanese Patent No. J60019-754-A and Hungarian Patent HU 200 996 B, which are herein incorporated by reference. One preferred compound comprises Avenanthramide C (Av-C).

In another embodiment, the present invention can be used as a nutraceutical formulation, additive, or supplement. In one embodiment, the present invention could be used to produce supplements containing extracted and purified phenolic compounds. In a preferred embodiment, the food supplement would comprise purified avenanthramides. In another embodiment, the oat alcoholic extract and/or avenanthramides could be incorporated into nutritional supplements that may be added to one's diet for beneficial health effects.

The proliferation of intimal vascular smooth muscle cells (SMC) and impaired NO production are both crucial pathophysiological processes in the initiation and development of atherosclerosis. In one aspect, the methods of the invention can be used to increase NO production. The invention discloses that substantially purified phenolic compounds can increase NO production in both human aortic smooth muscle cells (HSMC) and human aortic endothelial cells (HAEC). The phenolic compounds of the present invention can also increase expression of endothelial nitric oxide synthase (eNOS) by SMC and endothelial cells. In another embodiment, methods of the present invention can be used to reduce blood pressure through increasing NO.

In another aspect, methods of inhibiting proliferation of human aortic smooth muscle cell (HSMC) and/or human vascular smooth muscle cells (VSMC) through the administration of the phenolic compounds of the present invention. The method further comprises up-regulating the p53-p21cip1 pathway. Up-regulation of the p53-p21cip1 pathway can result in inhibition of pRB phosphorylation. In a preferred embodiment, the methyl ester form of the phenolic compounds can be used. Increased potency on the inhibition of cell proliferation can be achieved with the methyl ester form of the phenolic compounds as compared to the non-methylated forms.

The methods of the invention can be used in the reduction, treatment or prophylaxis of conditions caused by modified production of nitric oxide and/or modulated proliferation of VSMC. Non-limiting examples of such conditions include cardiovascular diseases, pathological platelet aggregation, pathological vasoconstriction, vascular effects of diabetes, stroke, atherosclerosis, hypertension, abnormal vasospasm, and restenosis after angioplasty. In one embodiment, a pharmaceutical composition could be made comprising a phenolic composition, or methyl ester form thereof, a pharmaceutically acceptable salt thereof, and a pharmaceutically acceptable carrier or diluent.

In one embodiment, the methods of the present invention are useful in modulating atherosclerosis and restenosis after angioplasty. VSMC proliferate significantly during atherosclerosis which contributes to the development of atheroma lesions in arteries, and can eventually lead to a narrowing of arteries and/or a heart attack. In one embodiment, the compounds of the present invention can be directly applied to target tissue, such as, for example, through intravenous administration or intraperitoneal injection. In another embodiment, the compounds of the present invention can be incorporated onto a variety of stents to prevent proliferation of SMC and/or prevent restenosis following application of stents. For example, the phenolic compounds of the present invention can be incorporated into the polymer resin of a stent, a drug eluting stent, or biodegradable stent, or incorporated into a polymer coating of the stent. The stent can then be used, for example, at the site of the narrowed artery through angiography procedure to reduce cell adhesion to polymer and modulate cell proliferation. The phenolic compounds can be incorporated into any polymeric resin, such as, for example, poly (L-lactic acid) (PLLA).

The methods of the invention can also be used to decrease cell expression of adhesion molecules, production of chemokines and pro-inflammatory of cytokines. Endothelial cells do not normally express elevated levels of adhesion molecules and thus, do not normally support excessive attachment to leukocytes. However, such an interaction is stimulated by exposure to a number of stimuli, including LDL, oxidized LDL, bacterial lipopolysaccharide, and inflammatory cytokines such as IL-1β, which induces phenotypic changes. The cytokine- and/or chemokine-stimulated adhesion of monocytes to endothelium of bovine, porcine, and human origin has been routinely used as a model to investigate interactions between leukocytes and the endothelium. Pretreatment of HAEC with oat extracts dose-dependently reduced IL-1β stimulated HAEC expression of adhesion molecules, productions of chemokines and pro-inflammatory of cytokines and their adherence to U937 cells.

The methods of the invention can be used in the reduction, treatment or prophylaxis of conditions caused by modified production and/or secretion of pro-inflammatory molecules or cell adhesion molecules. Non-limiting examples of such conditions include inflammatory conditions, free radical associated disorders, pain, autoimmune diseases, cardiovascular diseases, and atherosclerosis. In one embodiment, a pharmaceutical composition could be made comprising a phenolic composition, or a pharmaceutically acceptable salt thereof, and a pharmaceutically acceptable carrier or diluent. In another embodiment, the phenolic composition could be used as a topical lotion to treat inflammatory conditions.

The methods of the present invention represent a significant improvement over readily available treatment of cardiovascular disease and inflammation. Since leukocyte adhesion to the endothelium occurs early in the pathogenesis of atherosclerosis and inflammation, the methods of the present invention may be used, for example, to prevent new lesions or atherosclerotic plaques from forming, as well as enabling previously developed lesions to regress. Therefore, the present invention has the possibility to cure the disease or prevent its occurrence, which is a vast improvement over the currently available treatments that simply attempt to slow disease progression.

In another aspect, the present invention features methods and formulations or compositions for the treatment of colon cancer, and particularly for the inhibition, prevention and/or reduction of cancerous cell growth, as well as the destruction of early stage cancerous cells within the colon region of a mammal, wherein the formulations and compositions comprise an identified amount or effective concentration of one or more phenolic compounds or an active ingredient purified from oats. This invention also provides a method of treating or inhibiting colonic polyps or treating or inhibiting colorectal cancer in a subject in need thereof which comprises administering to the subject one or more phenolic compounds in combination with another agent. Exemplary agents that can be combined with the phenolic compounds of the present invention include, but are not limited to, vitamin E, B vitamins, zinc, selenium, copper, iron, manganese, magnesium, β-carotene, and other phytochemicals, NSAIDs, and polyphenols (such as resveratrol, curcumin, and EGCG). The NSAID can be selected from the group consisting of ibuprofen, sulindac, ketoporfen, fenoprofen, flurbiprofen, naproxen, tiaprofenic acid, suprofen, etodolac, carprofen, ketrolac, piprofen, indoprofen, celecoxib, rofecoxib, mobicox, and benoxaprofen. In some embodiments, COX-2 production can be decreased by approximately 20%, 30%, 40%, 50%, 60%, 70%, 80%, or 90% compared to normal production levels.

### BRIEF DESCRIPTION OF THE DRAWINGS

FIG. 1 is a bar graph showing oat extracts and DMSO cytotoxicity on confluent human aortic endothelial cells (HAEC);

FIG. 2 is a bar graph comparing the effects of 24 h incubation with varying concentrations of oat extracts on monocyte-endothelial cell adhesion;

FIG. 3A is a bar graph comparing the effects of 24 h incubation with varying concentrations of oat extracts on HAEC expression of the adhesion molecule intracellular adhesion molecule-1 (ICAM-1);

FIG. 3B is a bar graph comparing the effects of 24 h incubation with varying concentrations of oat extracts on HAEC expression of the adhesion molecule vascular cell adhesion molecule-1 (VCAM-1);

FIG. 3C is a bar graph comparing the effects of 24 h incubation with varying concentrations of oat extracts on HAEC expression of the adhesion molecule endothelial leukocyte adhesion molecule-1 (E-selectin);

FIG. 4A is a bar graph comparing the effects of 24 h incubation with varying concentrations of oat extracts on constitutive HAEC expression of interleukin-8 (IL-8);

FIG. 4B is a bar graph comparing the effects of 24 h incubation with varying concentrations of oat extracts on IL-1β stimulated HAEC expression of IL-8;

FIG. 5 is a bar graph comparing the effects of 24 h incubation with varying concentrations of oat extracts on IL-1β stimulated HAEC expression of IL-6;

FIG. 6A is a bar graph comparing the effects of 24 h incubation with varying concentrations of oat extracts on constitutive HAEC expression of monocyte chemoattractant protein-1 (MCP-1);

FIG. 6B is a bar graph comparing the effects of 24 h incubation with varying concentrations of oat extracts on IL-1β stimulated HAEC expression of MCP-1;

FIG. 7 is a bar graph showing that treatment of human aortic smooth muscle cells (HSMC) with avenanthramides inhibits FBS-induced DNA synthesis;

FIG. 8A is a graph showing that phenolic compounds of the present invention inhibit proliferation of human aortic smooth muscle cells (HSMC);

FIG. 8B is a graph showing that phenolic compounds of the present invention inhibit proliferation of A10 cells (rat embryonic aortic smooth muscle cells);

FIG. 9A is a bar graph showing that phenolic compounds of the present invention dose-dependently increased NO production of human aortic smooth muscle cells (HSMC);

FIG. 9B is a bar graph showing that phenolic compounds of the present invention dose-dependently increased NO production of human aortic endothelial cells (HAEC);

FIG. 10A is a bar graph showing that phenolic compounds of the present invention dose-dependently increased eNOS mRNS expression of human aortic smooth muscle cells (HSMC) as detected by real time PCR;

FIG. 10B is a bar graph showing that phenolic compounds of the present invention dose-dependently increased eNOS mRNA expression of human aortic endothelial cells (HAEC) as detected by real time PCR;

FIG. 11 is a Western blot demonstrating that phenolic compounds of the present invention inhibit pRb phosphorylation in A10 cells stimulated with FBS;

FIG. 12 is a Western blot demonstrating that phenolic compounds of the present invention inhibit FBS-induced cyclin D1 expression in A10 cells;

FIG. 13 is a Western blot demonstrating that phenolic compounds of the present invention increase p53 protein expression by increasing the stability of p53 protein in A10 cells that were seeded into 100 mm Petri dishes and grown in 10% FBS culture medium until 70% confluence and then treated with different concentrations of Avn for 24 h;

FIG. 14 is a Western blot demonstrating that phenolic compounds of the present invention increase p21cip expression level but has no significant effect on p27kip expression level;

FIG. 15 is a Western blot demonstrating that phenolic compounds of the present invention increase p53 protein expression by increasing the stability of p53 protein in A10 cells that were pretreated with 80 μM Avn for 6 h, then treated with cycloheximide (15 μg/mL);

FIG. 16 is a schematic of the chemical structure of phenolic compounds of the present invention;

FIG. 17 is a bar graph showing the effect of CH₃-Avn-C on FBS-induced DNA synthesis of A10 cells;

FIG. 18 is a bar graph showing the effect of CH₃-Avn-C on cell growth of A10 cells;

FIG. 19 is a bar graph showing the effect of CH₃-Avn-C o on reactive oxygen species (ROS) production of monocytic cells;

FIGS. 20A-D are bar graphs showing that phenolic compounds inhibit cell proliferation of growing phase Caco2 (A and C) cells but have little effects on differentiated Caco2 cells (B and D);

FIG. 21A-F are bar graphs showing that phenolic compounds inhibit proliferation of three different colon cancer cell lines, HT29 (FIG. 21A&B), LS174 (FIG. 21C&D), and HCT116 (FIG. 21E&F);

FIG. 22 is a Western blot showing that phenolic compounds inhibited TNF-α stimulated COX2 expression in HT29 colon cancer cells;

FIG. 23 is a Western blot showing that phenolic compounds have no effect on COX1 expression in HT29 colon cancer cells;

FIG. 24 is a bar graphs showing that phenolic compounds inhibit PGE2 levels in HT29 cell culture medium stimulated with TNF-α;

FIG. 25 is a Western blot showing that phenolic compounds inhibit the NFkB phosphorylation of HT29 colon cancer cells stimulated with TNF-α.

### DETAILED DESCRIPTION OF THE INVENTION

The present invention is directed to utilizing substantially purified phenolic compounds and/or oat extracts to increase NO production, to inhibit vascular smooth muscle cell proliferation and to modulate immune responses. The methods of the invention can be used to decrease production, secretion, and release of immune molecules such as pro-inflammatory molecules and cell adhesion molecules. The present invention can be used to treat, reduce, and/or prevent diseases resulting from modified immune responses including, but not limited to, inflammatory disorders, pain, autoimmune diseases, cardiovascular diseases, free radical associated disorder, and atherosclerosis.

So that the invention is more clearly understood, the following terms are defined:

The term “avenanthramide” as used herein refers to nitrogen containing phenolic compounds. The substituted N-cinnamolylanthranilate alkaloids occur naturally and can be purified from oat groats or hulls where they appear to be most concentrated in the peripheral regions. The avenanthramides comprise conjugated forms of aminophenolic acids, anthranilic, 5-hydroxyanthranilic, 4-hydroxanthranilic. Various hydroxy/methoxy substituted cinnamic or phenylpentadienoic acids attached via linkage to the amide of the aminophenolic moiety comprise the conjugated forms. More than 50 distinct avenanthramides have been isolated from oat grains.

The term “phenolic compounds” as used herein refers to a member of a class of organic molecules which have an aromatic ring with one or more hydroxyl substituents. These compounds comprise a wide spectrum of plant substances and frequently occur attached to sugars. Phenolic compounds, are powerful antioxidants that are found in potatoes, tomatoes, peppers, parsley, squash, yams, celery, carrots, cabbage, soybeans, flaxseed, whole grains, fruits, including citrus, some nuts and garlic. More than 200 phenolic compounds have been identified. Flavonoids, C15 compounds composed of two phenolic rings connected by a three-carbon unit, are the largest group of phenolic compounds.

Several phenolic compounds, such as avenanthramides, caffeic acid, ferulic acid, vanillic acid, sinapic acid, p-coumaric acid, and p-hydroxybenzoic acid flavonoids, have been identified in oats (Peterson, D. J. Cereal Sci. 33: 115-129 (2001)). From analytical and structural chemistry standpoints, these can be roughly divided into low molecular weights, readily soluble “free phenolics” (such as tocols, flavonoids, hydroxycinnamates, etc.), and “bound phenolics,” those covalently linked to complex high molecular weight, insoluble cell components (such as lignin, cell wall polysaccharides, structural protein, etc.). The “free phenolics” appear to represent readily absorbed sources of antioxidants in the human diet, while insoluble “bound phenolics,” requiring further metabolism before absorption from the gastrointestinal tract, present different challenges in attempts to evaluate the long-term efficacy of these compounds. Unlike other cereals however, oats contain a unique source of low molecular weight soluble phenolics, the avenanthramides, not present in other cereal grains, which exhibit potent antioxidant properties. These antioxidants constitute by far the major phenolic antioxidants present in the kernel. They occur in relatively high concentrations in the outer regions of the oat kernel, (e.g. bran and sub-aleurone layers) (Dimberg, L. H. et al. Cereal Chem. 70: 637-641 (1992)), although they are not restricted to these tissues.

The term “phenolic compounds” is intended to cover the class of compounds described by the core structure shown below: 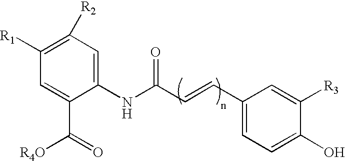
where n is less than or equal to six and R₁, R₂, and R₃ can be, but are not limited to a hydroxide, an aliphatic group, an aromatic group, an acyl group, an alkoxy group, an alkylene group, an alkenylene group, an alkynylene group, a hydroxycarbonylalkyl group, an anhydride, an amide, an amine, and a heterocyclic aromatic group, and R₄ is a hydrogen or an alkyl group. In some embodiments, n is less than three and the side chains are selected from the group consisting of H, OH, or OCH₃. In some embodiments, the phenolic compound is Avn-C, wherein n is 1, R₁ is OH, R₂ is hydrogen, R₃ is OH, and R₄ is hydrogen. In preferred embodiments, the alkyl ester form of the phenolic compounds can be used. In some embodiments, n is 1, R₁ is OH, R₂ is hydrogen, R₃ is OH, and R₄ is an alkyl group. The alkyl group is preferably a lower alkyl group which, for example, can be selected from the group consisting of methyl, ethyl, propyl, and butyl. Increased potency can be achieved with the alkyl ester form of the phenolic compounds as compared to the non-alkylated form of the phenolic compound.

The above structure is intended to include the various isomers that exist. Collins et al. (Collins et al. J. Chromatogr. 445: 363-370 (1988)) showed that phenolic compounds, such as avenanthramides, easily undergo Z-E rearrangement and that the E isomers appear to be more easily isolated. Isomers often can have very different activity. For example, Kakegawa and coworkers showed that the Z form of N-(3′,4′-dimethoxycin-namoyl)anthranilic acid has over 10 times the antiallergic activity as the E form.

The term “extract”, as used herein, is meant to encompass a compound or mixture of compounds that are obtained from oats. The extract can be obtained by extraction or distillation of any oat species, fresh or dried, or parts thereof, and is intended to incorporate any isomers that form or can form. Altering the composition of the solvent can change the extract composition, thus enhancing or reducing the biological activity. Work by Collins and co-workers resulting in U.S. Pat. No. 5,169,660 was able to show for the first time that phenolic compounds, known as avenanthramides, occur naturally and can be extracted from oat grain.

The terms “purified” and “substantially purified,” as used interchangeably herein, refer to a compound that is at least 60%, by weight, free from proteins and naturally-occurring organic molecules with which it is naturally associated. Preferably the preparation is at least 75%, more preferably 90%, and most preferably at least 99%, by weight, chemical compound, e.g., Avenanthramide C. A purified compound may be obtained, for example, by high pressure liquid chromatograph, thin layer chromatography, or by synthesizing it.

The term “aliphatic” as used herein refers to open-chain (non-cyclic) hydrocarbons. Aliphatic is especially used in reference to open-chain (non-cyclic) hydrocarbons. The term also refers to open-chain hydrocarbon sub-units of larger organic molecules. The aliphatic group may be further substituted by additional aliphatic or aromatic groups. Non-limiting examples of aliphatic groups consist of alkyl, alkenyl, alkynyl, cycloalkyl, cycloalkenyl, heterocyclyl, heterocyclenyl, aralkenyl, aralkyloxyalkyl, aralkyloxycarbonylalkyl, aralkyl, aralkynyl, aralkyloxyalkenyl, heteroaralkenyl, heteroaralkyl, heteroaralkyloxyalkenyl, heteroaralkyloxyalkyl, heteroaralkynyl, fused arylcycloalkyl, fused heteroarylcycloalkyl, fused arylcycloalkenyl, fused heteroarylcycloalkenyl, fused arylheterocyclyl, fused heteroarylheterocyclyl, fused arylheterocyclenyl, fused heteroarylheterocyclenyl, and the like. “Aliphatic”, as used herein, also encompasses the residual, non-carboxyl portion of natural and unnatural amino acids as defined herein.

The term “aromatic” as used herein refers to both aryl and heteroaryl rings. The aryl or heteroaryl ring may be further substituted by additional aliphatic or aromatic radicals. Representative aromatic groups include aryl, fused cycloalkenylaryl, fused cycloalkylaryl, fused heterocyclylaryl, fused heterocyclenylaryl, heteroaryl, fused cycloalkylheteroaryl, fused cycloalkenylheteroaryl, fused heterocyclenylheteroaryl, fused heterocyclylheteroaryl, and the like.

The term “acyl” as used herein refers to an H—CO— or alkyl-CO— group. Preferred acyl groups contain a lower alkyl, formyl, acetyl, propanoyl, 2-methylpropanoyl, butanoyl or palmitoyl.

The term “acylamino” as used herein refers to an acyl-NH- group.

The term “alkenyl” as used herein refers to a straight or branched aliphatic hydrocarbon group of 2 to about 15 carbon atoms which contains at least one carbon—carbon double bond. Preferred alkenyl groups have 2 to about 12 carbon atoms; more preferred alkenyl groups have 2 to about 4 carbon atoms. The alkenyl group may be substituted with one or more alkyl group substituents as defined herein. Representative alkenyl groups include ethenyl, propenyl, n-butenyl, i-butenyl, 3-methylbut-2-enyl, n-pentenyl, heptenyl, octenyl, cyclohexylbutenyl and decenyl.

The term “alkoxy” as used herein refers to an alkyl-O— group wherein the alkyl group is as defined herein. Representative alkoxy groups include methoxy, ethoxy, n-propoxy, i-propoxy, n-butoxy, heptoxy, and the like.

The term “alkyl” as used herein refers to an aliphatic hydrocarbon group, which may be straight or branched-chain, having about 1 to about 20 carbon atoms in the chain. Preferred alkyl groups have 1 to about 12 carbon atoms in the chain. Branched means that one or more lower alkyl groups such as methyl, ethyl or propyl are attached to a linear alkyl chain. “Lower alkyl” means 1 to about 4 carbon atoms in the chain, which may be straight or branched. The alkyl may be substituted with one or more “alkyl group substituents” which may be the same or different, and include halo, cycloalkyl, hydroxy, alkoxy, amino, carbamoyl, acylamino, aroylamino, carboxy, alkoxycarbonyl, aralkyloxycarbonyl, or heteroaralkyloxycarbonyl. Representative alkyl groups include methyl, trifluoromethyl, cyclopropylmethyl, cyclopentylmethyl, ethyl, n-propyl, i-propyl, n-butyl, 1-butyl, n-pentyl, 3-pentyl, methoxyethyl, carboxymethyl, methoxycarbonylethyl, benzyloxycarbonylmethyl, and pyridylmethyloxycarbonylmethyl.

The term “alkylene” as used herein refers to a straight or branched bivalent hydrocarbon chain of 1 to about 6 carbon atoms. The alkylene may be substituted with one or more “alkylene group substituents” which may be the same or different, and include halo, cycloalkyl, hydroxy, alkoxy, carbamoyl, carboxy, cyano, aryl, heteroaryl or oxo. Preferred alkylene groups are the lower alkylene groups having 1 to about 4 carbon atoms. Representative alkylene groups include methylene, ethylene, and the like.

The term “alkenylene” as used herein refers to a straight or branched bivalent hydrocarbon chain containing at least one carbon-carbon double bond. The alkenylene may be substituted with one or more “alkylene group substituents” as defined herein. Representative alkenylene include —CH═CH—, —CH₂CH═CH—, —C(CH₃)═CH—, —CH₂CH═CHCH₂—, and the like.

The term “alkynylene” as used herein refers to a straight or branched bivalent hydrocarbon chain containing at least one carbon—carbon triple bond. The alkynylene is optionally substituted with one or more “alkylene group substituents” as defined herein. Representative alkynylene include —C≡C—, —C≡C—CH₂—, —C≡C—CH(CH₃)—, and the like.

The term “alkynyl” used herein refers to a straight or branched aliphatic hydrocarbon group of 2 to about 15 carbon atoms which contains at least one carbon—carbon triple bond. Preferred alkynyl groups have 2 to about 12 carbon atoms. More preferred alkynyl groups contain 2 to about 4 carbon atoms. “Lower alkynyl” means alkynyl of 2 to about 4 carbon atoms. The alkynyl group may be substituted by one or more alkyl group substituents as defined herein. Representative alkynyl groups include ethynyl, propynyl, n-butynyl, 2-butynyl, 3-methylbutynyl, n-pentynyl, heptynyl, octynyl, decynyl, and the like.

The term “amino” used herein refers to a group of formula Z¹Z²N— wherein Z¹ and Z² are independently hydrogen; acyl; or alkyl, or Z¹ and Z² taken together with the N through which Z¹ and Z² are linked to form a 4 to 7 membered azaheterocyclyl. Representative amino groups include amino (H₂N—), methylamino, dimethylamino, diethylamino, and the like.

The term “aminoalkyl” used herein refers to an amino-alkylene- group wherein amino and alkylene are defined herein. Representative aminoalkyl groups include aminomethyl, aminoethyl, dimethylaminomethyl, and the like.

The term “aralkyl” used herein refers to an aryl-alkylene- group wherein aryl and alkylene are as defined herein. Preferred aralkyls contain a lower alkyl moiety. Representative aralkyl groups include benzyl, 2-phenethyl, naphthlenemethyl, and the like.

The term “aroyl” used herein refers to an aryl-CO— group wherein aryl is defined herein. Representative aroyl include benzoyl, naphth-1-oyl and naphth-2-oyl.

The term “cycloalkyl” used herein refers to a non-aromatic mono- or multicyclic ring system of about 3 to about 10 carbon atoms, preferably of about 5 to about 10 carbon atoms. Preferred cycloalkyl rings contain about 5 to about 6 ring atoms. The cycloalkyl can be substituted with one or more “ring system substituents” which may be the same or different, Representative multicyclic cycloalkyl include 1-decalin, norbornyl, adamantyl, and the like.

There term “cycloalkenyl” used herein refers to a non-aromatic mono- or multicyclic ring system of about 3 to about 10 carbon atoms, preferably of about 5 to about 10 carbon atoms which contains at least one carbon-carbon double bond. Preferred cycloalkylene rings contain about 5 to about 6 ring atoms. The cycloalkenyl may be substituted with one or more “ring system substituents” which may be the same or different, and are as defined herein. Representative monocyclic cycloalkenyl include cyclopentenyl, cyclohexenyl, cycloheptenyl, and the like. A representative multicyclic cycloalkenyl is norbornylenyl.

There term “aryl” used herein refers to an aromatic monocyclic or multicyclic ring system of 6 to about 14 carbon atoms, preferably of 6 to about 10 carbon atoms. The aryl may be substituted with one or more “ring system substituents” which may be the same or different, and are as defined herein. Representative aryl groups include phenyl and naphthyl.

The term “heteroaryl” used herein refers to an aromatic monocyclic or multicyclic ring system of about 5 to about 14 ring atoms, preferably about 5 to about 10 ring atoms, in which one or more of the atoms in the ring system is/are element(s) other than carbon, for example nitrogen, oxygen or sulfur. Preferred heteroaryls contain about 5 to about 6 ring atoms. The “heteroaryl” is optionally substituted by one or more “ring system substituents” which may be the same or different, and are as defined herein. The prefix aza, oxa or thia before heteroaryl means that at least a nitrogen, oxygen or sulfur atom respectively is present as a ring atom. A nitrogen atom of a heteroaryl is optionally oxidized to the corresponding N-oxide. Representative heteroaryls include pyrazinyl, furanyl, thienyl, pyridyl, pyrimidinyl, isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, imidazo[1,2-a]pyridine, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like.

The term “benzyl” used herein refers to a phenyl-CH₂— group. Substituted benzyl means a benzyl group in which the phenyl ring is substituted with one or more ring system substituents. Representative benzyl include 4-bromobenzyl, 4-methoxybenzyl, 2,4-dimethoxybenzyl, and the like.

The terms “carboxy” and “carboxyl” used herein refers to a HO(O)C— group (i.e. a carboxylic acid).

The term “carboxyalkyl” used herein refers to a HO(O)C-alkylene- group wherein alkylene is defined herein. Representative carboxyalkyls include carboxymethyl and carboxyethyl.

The term “cycloalkyloxy” used herein refers to a cycloalkyl-O— group wherein cycloalkyl is as defined herein. Representative cycloalkyloxy groups include cyclopentyloxy, cyclohexyloxy, and the like.

The term “hydroxyalkyl” used herein refers to an alkyl group as defined herein substituted with one or more hydroxy groups. Preferred hydroxyalkyls contain lower alkyl. Representative hydroxyalkyl groups include hydroxymethyl and 2-hydroxyethyl.

The term “ring system substituents” used herein refers to substituents attached to aromatic or non-aromatic ring systems inclusive of hydrogen, alkyl, aryl, heteroaryl, aralkyl, aralkenyl, aralkynyl, heteroaralkyl, heteroaralkenyl, heteroaralkynyl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylsulfinyl, arylsulfinyl, heteroarylsulfinyl, alkylthio, arylthio, nitrile, NO₂ heteroarylthio, aralkylthio, heteroaralkylthio, cycloalkyl, cycloalkenyl, heterocyclyl, heterocyclenyl, aryidiazo, heteroaryldiazo, amidino, Z¹Z²N—, Z¹Z²N-alkyl-, Z¹Z²NCO— or Z¹Z²NSO₂—, wherein Z¹ and Z² are independently hydrogen, alkyl, aryl, and aralkyl, or where the substituent is Z¹Z²N— or Z¹Z²N-alkyl- then one of Z¹ and Z² is acyl or aroyl and the other of Z¹ and Z² is hydrogen, alkyl, aryl, and aralkyl. When a ring system is saturated or partially saturated, the “ring system substituent” further comprises methylene (H₂C═), oxo (O═) and thioxo (S═). Preferred ring system substituents are hydrogen, CF₃, fluoro, alkyl, alkoxy, nitrile or NO₂.

The term “inflammatory disorder” used herein refers to a response to a tissue injury caused by pathogenic microorganisms, trauma, chemicals, toxins, heat, or immune defenses (i.e. autoimmune diseases) involving secretion of several mediators from the injured tissue and induction of immunocytes. When tissue cells are damaged or destroyed, acids and chemical mediators (i.e. cytokines, histamine, bradykinin, serotonin, etc.) get released resulting in the dilation and increased permeability of blood capillaries. Histamine secreted from mast cells or basophiles initiates the response of blood vessels, and serum kinin produced from alpha-2-globulin of blood serum mediates the long-acting response of blood vessels through the blood coagulation mechanism. The blood capillary dilation increases the blood flow, and causes heat and redness. The increased permeability of the blood capillaries cause blood cells, proteins and fluids to exude into surrounding tissues, leading to swelling. Such exudation can accelerate further destruction of cells, and the increased blood pressure stimulates peripheral nerves to cause pain. The pain increases due to secretion of kinin and acids. Other mediators secreted from the tissue include serotonin, prostaglandin, reactants of the complement system, and lymphokine secreted from T-cells.

The inflammatory reaction can occur locally or become systemic. In some cases, pyrogens secreted from bacteria stimulate the thermoregulatory center in the brain and produce a fever. In addition, inflammatory disorders comprise autoimmune disorders which are diseases caused by the body producing an immune response against its own tissues. Autoimmune disorders can be classified into two groups: systemic, causing damage to many organs, and localized, where only a single organ or tissue is directly damaged. Non-limiting examples of autoimmune disorders, with the organ affected include Hashimoto's thyroiditis and Graves' disease (thyroid gland); pernicious anemia (stomach); Addison's disease (adrenal glands); Celiac disease, Crohn's disease, and ulcerative colitis (GI tract); multiple sclerosis and Guillain-Barre syndrome (brain); primary biliary sclerosis, sclerosing cholangitis, and autoimmune hepatitis (liver); and insulin-dependent diabetes mellitus (pancreas). Examples of autoimmune disorders in which multiple organs are affected include, but are not limited to, rheumatoid arthritis, systemic lupus erythematosus (SLE or lupus), Scleroderma, Sjogren's syndrome, Goodpasture's syndrome, Wegener's granulomatosis, and dermatomyositis.

The term “cytokine” as used herein refers to a member of a group of soluble (glyco)proteins released by a cell to send messages and that can act on the same cell (autocrine), on an adjacent cell (paracrine), or on a distant cell (endocrine). Upon binding to a specific receptor, the cytokine causes a change in function or in development of the target cell. Cytokines are involved in reproduction, growth and development, normal homeostatic regulation, response to injury and repair, blood clotting, and host resistance (immunity). Cytokines are produced by many different cell types which modulate the function of other cell types. Cytokines play a role in cellular immune responses as well as inflammatory responses. Cytokines can be classified into five groups: interleukins, colony-stimulating factors, interferons, tumor necrosis factors, and growth factors. Interleukins (IL) is a term used for cytokines produced by leukocytes or macrophage and that act on another leukocyte. Specific interleukins are defined by their number. The main cytokines that mediate inflammation are IL-1, TNF, and IL-8.

Interleukin-1 is produced by macrophages, skin epithelium, fibroblasts, blood vessel endothelium, joint lining cells, cartilage cells, bone-marrow and circulating leukocyte cells, liver cells, adrenal gland cells, pancreatic islet beta cells, fertilized eggs, and cells in the nervous system. IL-1 binds to receptors on a wide variety of cells, including thymus-derived lymphocytes (stimulating immunity), those of the nervous system (causing fever), and endothelial cells (perhaps activating inflammation and blood clotting). Interleukin-6 is also known as beta-2 interferon, IFN-β2, and liver cell growth-stimulating factor, BSF-2, and BCDF. It is produced by a wide variety of cell types, and stimulates the liver to produce proteinase inhibitors and, thereby, dampens inflammation. Interleukin-8 is a group of peptides produced by a variety of cell types. They activate and recruit polymorphonuclear leukocytes in the inflammatory process, and may be involved in initiation of labor and delivery in pregnant women. Interleukin-10 is produced by a subset of helper T cells as well as by B lymphocytes and some cells of the uterus during pregnancy. It inhibits secretion (and function) of cytokine by macrophages and the second population of helper T cells called Th1. The Th1 cells are pro-inflammatory and promote delayed-type hypersensitivity, and the generation of cytotoxic T lymphocytes (antigen-specific killers) and cytotoxic macrophages (nonspecific) in preference to immune responses giving rise to antibodies. Protection against lethal shock triggered by bacterial endotoxin is prevented by suppression of cytokine synthesis.

TNFα and IL-1β are mainly produced from mononuclear leucocytes in response to an endotoxin. They cause increased synthesis of each other and stimulate the production of IL-6, IL-8 and IL-10. TNFα and IL-1β produce fever, activate the clotting system and mediate inflammation through production of IL-8 and by stimulating expression of adhesion molecules. IL-6 stimulates production of acute phase proteins from the liver and acts to inhibit the production of TNFα and IL-1β. Expression of inflammatory cytokines from their respective genes is controlled by intracellular transcription factors in particular nuclear factor kappa B (NFκB). NFκB is a primary transcription factor pre-existent in the cellular cytoplasm complexed with the inhibitory subunit IκB. In response to extracellular stimuli, IκB undergoes phosphorylation and ubiquitination allowing its proteosomal degradation. Free NFκB is able to translocate into the nucleus and bind to the promoter region of its target gene

The terms “modifies,” “modified,” and “modulate” are used interchangeably herein and refer to the increase, decrease, elevation, or depression of processes or signal transduction cascades resulting in the altered production or secretion of a protein, peptide, or secondary messenger. The term modifies or modified also refers to the up-regulation or down-regulation of a target gene or a target protein. Non-limiting examples of modifications includes modifications of morphological and functional processes, under- or over production or expression of a substance or substances, e.g., a cytokine, by lymphocytes, failure of cells to produce a substance or substances which it normally produces, production of substances. This modification can result in a variety of disease states.

As used herein, a “therapeutic composition” refers to a composition comprising an active ingredient required to cause a desired effect when a effective amount of the composition is administered to a subject in need thereof.

Within the present invention, an “effective amount” of a composition is that amount of each active component of the therapeutic composition that is sufficient to show a benefit (e.g., a reduction in a symptom associated with the disorder, disease, or condition being treated, or an increase in NO production, decrease in smooth muscle cell proliferation, or decrease in pro-inflammatory and/or cell adhesion molecules). Sample assays to verify the effect are described in the Examples section. When applied to an individual active ingredient, administered alone, the term refers to that ingredient alone. When applied to a combination, the term refers to combined amounts of the active ingredients that result in the benefit, whether administered in combination, serially, or simultaneously.

The term “immune response” as used herein refers to any change in a subject that defends against microorganisms, cancer, disease, or other potentially harmful substances. The response can be cell mediated or antibody mediated and results in change in the production or secretion of cytokines, white blood cells (i.e., neutrophils, lymphocytes (B and T cells), macrophages), chemicals and/or proteins. An immune response includes but is not limited to an inflammatory response, complement mediated, acquired or adaptive immunity, or passive immunity. Immune system disorders occur from inappropriate, excessive, or lacking immune responses. Allergies involve an immune response to a substance that, in the majority of people, the body perceives as harmless. Transplant rejection involves the destruction of transplanted tissues or organs and is a major complication of organ transplantation. Blood transfusion reaction is a complication of blood administration. Autoimmune disorders (such as systemic lupus erythematosus and rheumatoid arthritis) occur when the immune system acts to destroy normal body tissues. Immunodeficiency disorders, such as inherited immunodeficiency and AIDS, occur when there is a failure in all or part of the immune system.

The term “adhesion molecules” or “cell adhesion molecules (CAMs)” as used interchangeably herein, refer to cell surface proteins involved in the binding of cells. The cells, usually leukocytes, can be bound to each other, to endothelial cells, or to extracellular matrix. Specific signals triggered in response to injury and infection control the expression and activation adhesion molecules. Adhesion molecules, following binding to their receptors/ligands, play important roles in the mediation of the inflammatory and immune reactions that comprise one group of the body's defense against insults. Most adhesion molecules characterized so far fall into three general families of proteins: the immunoglobulin (Ig) superfamily, the integrin family, or the selectin family. The members of the Ig superfamily, such as ICAM-1, ICAM-2, ICAM-3, VCAM-1, and MadCAM-1, bind to integrins on leukocytes and mediate their flattening onto the blood vessel wall with their subsequent extravasation into the surrounding tissue. Chemokines such as MCP-1 and IL-8 cause a conformational change in integrins so that they can bind to their ligands. The integrin family act as receptors for the ICAMs and VCAMs. The integrins are heterodimeric proteins consisting of an alpha and a beta chain that mediate leukocyte adherence to the vascular endothelium or other cell-cell interactions.

Different groups of integrins are expressed by different populations of leukocytes providing specificity for binding to different types of adhesion molecules expressed along the vascular endothelium. The selectin family members, L-Selectin, P-Selectin, and E-Selectin, are involved in adhesion of leukocytes to activated endothelium, which is initiated by weak interactions that produce a characteristic “rolling” motion of the leukocytes on the endothelial surface and lead to extravasation through the blood vessel walls into lymphoid tissues and sites of inflammation.

Tissue injury occurs during inflammation and is a progressive process which may eventually lead to organ dysfunction and failure. Circulating neutrophils interact with the vascular endothelium in a three-stage process of rolling, adhesion and migration so that their normally rapid flow through the circulation can be diverted. Leukocyte rolling is mediated through pro-inflammatory cytokines induced expression of selectins on leucocytes and endothelium. Adhesion occurs through binding of leukocyte β2 integrins to endothelial intracellular adhesion molecule-1 (ICAM-1). Expression of adhesion molecules is increased in the most severely ill patients. Adherent leucocytes are then able to migrate into the tissues.

The term “antioxidant” as used herein refers to a substance that, when present in a mixture or structure containing an oxidizable substrate molecule (e.g., an oxidizable biological molecule or oxidizable indicator), significantly delays or prevents oxidation of the oxidizable substrate molecule. Antioxidants can act by scavenging biologically important reactive free radicals or other reactive oxygen species (e.g., O₂⁻, H₂ O₂, HOCl, ferryl, peroxyl, peroxynitrite, and alkoxyl), or by preventing their formation, or by catalytically converting the free radical or other reactive oxygen species to a less reactive species. Antioxidants can be separated into two classes, lipid antioxidants, and aqueous antioxidants. Examples of lipid antioxidants include, but are not limited to, carotenoids (e.g. lutein, zeaxanthin, β-cryptoxanthin, lycopene, α-carotene, and β-carotene), which are located in the core lipid compartment, and tocopherols (e.g. vitamin E, α-tocopherol, γ-tocopherol, and δ-tocopherol), which are located in the interface of the lipid compartment, and retinoids (e.g. vitamin A, retinol, and retinyl palmitate) and fat-soluble polyphenols such as quercetin. Examples of aqueous antioxidants include, but are not limited to, ascorbic acid and its oxidized form, “dehydroascorbic acid”, uric acid and its oxidized form, “allantoin”, bilirubin, albumin and vitamin C and water-soluble polyphenols such as catechins, which have high affinity to the phospholipid membranes, isoflavones, and procyanidins.

The term “free radical” as used herein refers to molecules containing at least one unpaired electron. Most molecules contain even numbers of electrons, and their covalent bonds normally consist of shared electron pairs. Cleavage of such bonds produces two separate free radicals, each with an unpaired electron (in addition to any paired electrons). They may be electrically charged or neutral and are highly reactive and usually short-lived. They combine with one another or with atoms that have unpaired electrons. In reactions with intact molecules, they abstract a part to complete their own electronic structure, generating new radicals, which go on to react with other molecules. Such chain reactions are particularly important in decomposition of substances at high temperatures and in polymerization. In the body, oxidized (see oxidation-reduction) free radicals can damage tissues. Antioxidant nutrients (e.g., vitamins C and E, selenium, polyphenols) may reduce these effects. Heat, ultraviolet light, and ionizing radiation all generate free radicals. Free radicals are generated as a secondary effect of oxidative metabolism. An excess of free radicals can overwhelm the natural protective enzymes such as superoxide dismutase, catalase, and peroxidase. Free radicals such as hydrogen peroxide (H₂O₂), hydroxyl radical (HO.), singlet oxygen (¹O₂), superoxide anion radical (O.₂⁻), nitric oxide radical (NO.), peroxyl radical (ROO.), peroxynitrite (ONOO⁻) can be in either the lipid or compartments.

The phrase “free radical associated disorder” as used herein refers to a pathological condition in a subject that results at least in part from the production of or exposure to free radicals, for example, oxyradicals, or other reactive oxygen species in vivo. The term “free radical associated disorder” encompasses pathological states that are recognized in the art as being conditions wherein damage from free radicals is believed to contribute to the pathology of the disease state, or wherein administration of a free radical inhibitor (e.g., desferrioxamine), scavenger (e.g., tocopherol, glutathione), or catalyst (e.g., SOD, catalase) are shown to produce a detectable benefit by decreasing symptoms, increasing survival, or providing other detectable clinical benefits in protecting or preventing the pathological state. Examples of free radical disorders include, but are not limited to, ischemic reperfusion injury, inflammatory diseases, systemic lupus erythematosis, myocardial infarction, stroke, traumatic hemorrhage, spinal cord trauma, Crohn's disease, autoimmune diseases (e.g., rheumatoid arthritis, diabetes), cataract formation, age-related macular degeneration, Alzheimer's disease, uveitis, emphysema, gastric ulcers, oxygen toxicity, neoplasia, undesired cell apoptosis, and radiation sickness. Such diseases can include “apoptosis-related ROS” which refers to reactive oxygen species (e.g., O₂⁻) which damage critical cellular components (e.g., lipid peroxidation) in cells stimulated to undergo apoptosis, such apoptosis-related ROS which may be formed in a cell in response to an apoptotic stimulus and/or produced by non-respiratory electron transport chains (i.e., other than ROS produced by oxidative phosphorylation).

The term “oxidative stress” as used herein refers to the level of damage produced by oxygen free radicals in a subject. The level of damage depends on how fast reactive oxygen species are created and then inactivated by antioxidants.

The phrase “inhibiting a condition associated with a lack of or need for nitric oxide (NO)” includes prohibiting, preventing, restraining, and slowing, stopping, or reversing progression, severity or a resultant symptom or effect of the physiological condition. Such conditions include those mentioned in this application, such as pathological platelet aggregation, pathological vasoconstriction, vascular effects of diabetes, stroke, atherosclerosis, hypertension, abnormal vasospasm, and restenosis after angioplasty.

The term “subject” as used herein refers to any living organism in which an immune response is elicited. The term subject includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered.

The invention is described in more detail in the following subsections:

I. Oat Species and Extraction Methods

Oats (called Avena in Latin) comprise a group of species that have large, drooping flowerheads and stout, twisted, bent awns growing from the back of the lemmas. Oats are believed to be derived chiefly from two species, wild oat (A. fatua L.) and wild red oat (A. sterilis L.). Oat species with different ploidy levels include but are not limited to: diploids (Avena pilosa, A. clauda, A. ventricosa, A. longiglumis, A. canariensis, A. hirtula, A. wiestii, A. atlantica), tetraploids (A. barbata, A. vaviloviana, A. magna, A. murphyi) and hexaploids (A. fatua, A. occidentalis, A. ludoviciana, A. sterilis).

Avena fatua or wild oat is an introduced species that grows to 80 cm tall. It is an annual with large, open, drooping flowerheads. Wild Oat was introduced from Eurasia. It occurs most often on waste ground, and is a weed in grain fields. In the Columbia Basin region it grows at Creston and Yoho National Park.

Avena sativa or common oat is an introduced species that grows to 80 cm tall. It is an annual with large, open, drooping flowerheads. Common Oat grows on roadsides, railways and waste places. Introduced from Eurasia, it does not persist as an escape from cultivation for more than a year. In the Columbia Basin region it was only collected at Kokanee Glacier Park.

The phenolic compounds can be extracted using known techniques for isolating such compounds from samples of oats, wheat and the like. For example, the phenolic compounds and isomers thereof can be extracted from any oat species, fresh or dried, or parts of oat species, e.g., the hull. Typically, phenolic compounds can be extracted using different solvents. Altering the composition of the solvent can change the extract composition, thus enhancing or reducing the biological activity of phenolic compounds. For example, avenanthramides extracted from oat grain have been described by Canadian patent 1,179,189, which teaches an aqueous steeping method, and U.S. Pat. No. 5,169,660. Isolation of avenanthramides from oat extract are described in Example 1. Three major groups of phytochemicals were separated based on their relative polarity, ranging from most fat soluble, lipophilic, to most water soluble, aqueous. The three groups are alkyl esters, avenanthramides, and oat flavonoids, these phenolics can be separated by liquid chromatography using acidified ethanol.

The isolating the avenanthramides involves the separation of the lipophilic phenolics from the hydrophilic phenolics, which is then further fractionated by double ion exchange, and analyzed by HPLC to yield at least 25 individual compounds. The hydrophilic phenolics can be then further fractionated to yield an avenanthramides fraction, comprising more than 40 different compounds. Identification and quantitation can be performed, for example, by HPLC spectroscopy. The typical oat groat profile consists of three predominant Avenanthramides, A, B and C and a number of isomers and extended chain analogs ((Peterson, D. M. J. Cereal Sci. 33: 115-129 (2001); (Dimberg et al., Cereal Chem., 70(6): 637-641 (1993)). These Avenanthramides can also be made synthetically.

II. Health Benefits of Phenolic Compound Compositions

Like all plant foods, oats contain biologically active chemicals or phytochemicals that have been recognized for their health benefits. Oats are also rich in antioxidants, and in particular, phenolic compounds, a class of antioxidants that includes avenanthramides, caffeic acid, ferulic acid, sinapic acid, and cinnamic acid (Collins, J. Agric. Food Chem. 37: 60-66 (1989)). The avenanthramides, a unique source of low molecular weight soluble phenolics present in oats, but not in other cereal grains, exhibit potent antioxidant properties and constitute by far the major phenolic antioxidants present in the oat kernel. Avenanthramides may be useful as antihistaminic, antiallergic, and antiasthmatic drugs, and as inhibitors of lipoxygenase.

In one aspect, the method of the invention relates to providing protection against initiation and development of heart disease, such as atherosclerosis, stroke, and hypertension. The invention can be used to reduce or prevent vascular dysfunction and development of atherosclerotic lesions. This invention can be used to reduce the risk of developing atherosclerosis and hypertension.

The proliferation of vascular smooth muscle cells (SMC) plays a pivotal role in the development and progression of atherosclerosis and restenosis following angioplasty. Vascular SMC comprises the medial layer of the vascular wall. Whereas the intimal layer of the artery contains a few SMC, which are scattered within the intimal extracellular matrix. Under normal conditions, SMC are maintained in a quiescent and contractile state. Whereas, during the chronic inflammation of artery, with the accumulation of foam cells in injured blood vessels and the continuous production of pro-inflammatory cytokines, the fatty streaks evolves into more complicated atheroma lesions, where SMC undergo a phenotypic transformation from a quiescent contractile state to a more synthetic phenotype and become activated, proliferated, and migrated to the intimal layer causing intimal thickening of the arterial wall. These changes in the characteristics of SMC also occur during restenosis after angioplasty or following the application of a stent in an artery, which may elicit inflammatory reactions. These conditions hitherto are major problems limiting long-term clinical outcome of these procedures in approximately 20-40% of the cases. Therefore, in addition to lowering blood cholesterol and triglyceride levels, inhibition of arterial SMC proliferation are considered to be of great benefit in the maintenance of vascular homeostasis and in the prevention of developing atherosclerosis as and restenosis following angioplasty or stent application. In one aspect, the invention provides methods for inhibiting SMC proliferation.

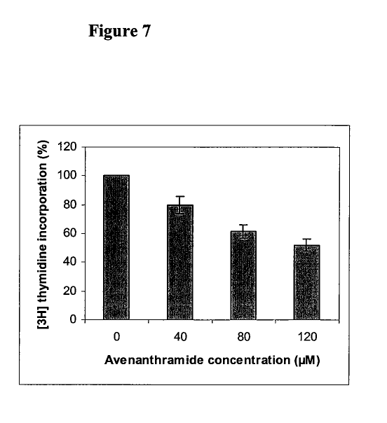

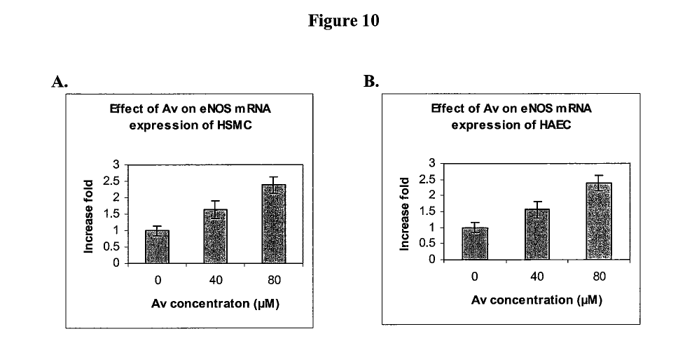

The invention provides methods of inhibiting vascular SMC proliferation. In another aspect, the invention provides methods of increasing NO production. The proliferation of intimal vascular smooth muscle cells (SMC) and impaired NO production are both crucial pathophysiological processes in the initiation and development of atherosclerosis. Phenolic compounds of the present invention can be used to decrease human aortic SMC (HASMC) proliferation and increase NO production (See Example 6 and 7). Avn-C, is one of the three major Avns found in oats and has the highest antioxidant activity in vitro. Avn-C can dose-dependently inhibit serum-induced HASMC proliferation as measured by [³H] thymidine incorporation and by counting cell numbers (See FIG. 7). The IC₅₀ of Av-C was around 50 μM. Incubation of cells with 120 μM Av-C for 4 days inhibited cell growth by more than 50%. This inhibitory effect of Av-C was associated with an increase in the expression of p21^{CIPI}, a cyclin-dependent kinase (CDK) inhibitor. In addition, Av-C treatment significantly increased NO production and eNOS mRNA expression as measured by real time PCR (See FIG. 10A&B). At 80 μM Av-C, NO production and eNOS mRNA expression levels increased by 2.1 and 3.5 fold, respectively. The HASMC expression of vascular cell adhesion molecular-1 (VCAM-1) was not affected by Av-C treatment. Thus, phenolic compounds of the present invention have been shown to inhibit the human aortic endothelial cells (HAEC) expression of adhesion molecules and their adhesion to monocytes and reduce production of several inflammatory cytokines and chemokines that are important in the development of atherosclerosis. In addition, the Examples demonstrate that fetal bovine serum (FBS)-induced cell proliferation of vascular SMC dose dependently was inhibited by synthetically prepared Avn-C.

The Examples demonstrate the mechanism by which the phenolic compounds of the present invention inhibits proliferation of SMC. Flow cytometry analysis revealed that treatment of A10 cell with 80 μM Avn arrested the cell cycle in G1 phase as indicated by the increase of the cell population in G1 phase and decrease of the cell number in S phase. This cell cycle arrest was associated with a decreased in the phosphorylation of retinoblastoma protein (pRb), whose hyperphosphorylation is a hallmark of the G1-S transition in the cell cycle. This inhibition of pRb phosphorylation with Avn was accompanied with a decrease in cyclin D1 expression, and an increase in cyclin-dependent kinase inhibitor p21cip1 expression, without a significant changes in p27kip1 expression. Furthermore, Avn treatment increased p53 protein expression level and its stability from, which could account for the increase of p21cip1 expression. The results demonstrate for the first time that Avn-c, which is present in oats inhibits SMC proliferation at G1 phase by up-regulating p53-p21cip1 pathway and inhibiting pRB phosphorylation. This inhibitory effect of Avn on SMC proliferation is another indication that the consumption of Avenanthramides is beneficial for prevention of cardiovascular disease.

In one aspect, the invention provides methods of inhibiting cell proliferation through the administration of the phenolic compounds of the present invention. The substantially puritied compounds modulate cell cycle regulating molecules, such as cyclin D₁, pRB, P21, and P27, resulting in inhibition of cell proliferation. Example 9 shows that treatment with Avn, such as Avn-C, prevents rat aortic smooth muscle cell (A10) cell cycle at the G1 phase. The product of the retinoblastoma tumor suppressor gene, pRB, is a major negative regulator of cell proliferation and operates in the middle to late G1 phase of the cell cycle. The pRB phosphorylation is brought about by a group of serine/threonine kinases, cyclin-dependent kinase (CDK). Cyclin D is expressed upon mitogen stimulation and associates with CDK to form active cyclin D/CDK complex, which is responsible for the phosphorylation of pRB in G1 phase. As shown in Example 9, Avn dose-dependently inhibits pRB phosphorylation. Since cyclin D upregulates CDK activity, the effect of Avn on serum-stimulated expression of cyclin D1 was explored. Example 10 shows that Avn treatment can attenuate FBS-induced cyclin D expression in smooth muscle cells, and this inhibitory effect is dose-dependent.

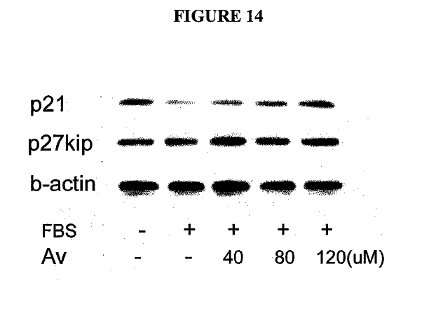

Induction of the tumor suppressor p53 has been implicated in the control of cell growth of SMC, and some antioxidants in green tea or red wine can increase the p53 protein expression level. As shown in Example 11, Avn produced a dose-dependent increase in p53 protein levels. P21cip1 is the main target of p53 medicated cell cycle inhibition. Upregulation of p21cip1 has also been shown to be one of the main mechanisms involved in the prevention of pRb phosphorylation, which causes cell cycle inhibition. As shown in FIG. 14, when quiescent A10 cells were stimulated with FBS in the presence of Avn, the p21 expression level increased significantly compared to control and this inducible effect of Avn was shown to be dose-dependent. In addition, as shown in Example 13, exposure of A10 cells to Avn causes an increase of half-life of p53. This increased protein stability can lead to the increased p53 levels induced by Avn treatment shown in Example 11. The Examples demonstrate that the phenolic compounds of the present invention can inhibit cell proliferation though the modulation of cell cycle regulating molecules, such as cyclin D1, pRB, P21, and P27.

In another aspect of the present invention, alkyl ester forms of the phenolic compounds were shown to inhibit vascular SMC proliferation. As shown in Examples 14, the methyl ester form of Avn-C is 10 times more potent than Avn-C.

In one aspect, the method of the invention relates to providing protection against free-radical induced disorders by administering phenolic compounds with antioxidant properties. Antioxidants can be characterized in different ways based upon their solubility, their mechanism, or their localization site within the body. Antioxidants can either be fat soluble (lipophilic), water soluble (hydrophilic) or both (Halliwell et al. Arch. Biochem. Biophys. 280:1-8 (1990)). Lipophilic antioxidants, such as carotenoids, can protect the cell membrane and enter the cell to protect other parts of the cell that are surrounded by lipid membranes. However, since it cannot dissolve in the blood, lipophilic antioxidants are transported attached to another molecule. Hydrophilic antioxidants, such as vitamin C, act in the blood. Since they cannot dissolve in the lipid membrane, they must be specifically transported into the cell where it can protect the aqueous parts of the cell. Some antioxidants, such as the avenanthramides, alpha lipoic acid and vitamin E, are both lipophilic and hydrophilic and hence can provide protection almost anywhere in the body. Antioxidants also differ in the class of free radicals (e.g. hydroxyl anion or singlet oxygen) that they can neutralize. For example, vitamin E is effective against peroxyl radicals, singlet oxygen, and peroxynitrite whereas carotenoids only protect against singlet oxygen or peroxyl radicals. Additionally, antioxidants can act as primary antioxidants, which decrease the initiation rate of peroxidation (i.e. transferrin and ceruloplasmin bind prooxidant metal ions) or as secondary antioxidants, which decrease the chain propagation and amplification of peroxidation (i.e. α-tocopherol scavenges oxidizing species). However, most antioxidants are not exclusive, but act with multiple antioxidant properties (e.g., uric acid).

Antioxidants also accumulate in and protect different parts of the body. For example, vitamin C accumulates in the lens of the eye providing protection from cataracts. The carotenoids β-carotene and lutein accumulate in the skin and protect it from the sun's damaging rays. Lutein also accumulates in the macula of the eye, reducing oxidative stress and the risk of macular degeneration. Vitamin E is absorbed into cell membranes, protecting them from oxidative stress. Coenzyme Q10 protects mitochondria from free-radical damage. Some bioflavonoids are thought to be important in protecting the integrity of blood vessels.

The method of the invention can be used to provide protection all over the body, i.e., in both the aqueous compartment and the lipid compartment. In another embodiment, the method of the invention relates to providing protection in a particular compartment, e.g., the lipid compartments or the aqueous compartment.

In another aspect of the invention, the phenolic compounds can be used to protect a subject from increased production or secretion of adhesion molecules that could lead to disease. In a preferred embodiment, avenanthramides could be administered to reduce risk for atherosclerosis or heart diseases. The protective effects of oat extract on inhibiting monocyte-HAEC adhesion is shown in Example 3. The protective effect of oat extract in suppressing expression of adhesion molecules is shown in Example 4.

The methods of the present invention could also be used to treat, reduce, or prevent inflammatory disorders. In another aspect of this invention, the composition could be used to treat or reduce inflammation caused by autoimmune disorders. Methods for producing topical and pharmaceutical compositions, which can be applied to the present invention, are described in U.S. Pat. No. 6,387,398 which is hereby incorporated by reference. The protective effect of oat extract by suppressing pro-inflammatory cytokines is shown in Examples 5.

The methods of the present invention can be used to maintain levels of physiologically acceptable inflammatory molecules in an individual. Administering oat extract can normalize levels of production or secretion of these molecules. Dosing ranges of the oat extract may vary dependent upon preparation. The composition or combination of agents can be administered in amounts sufficient to ensure that the serum level of the phenolic compounds is maintained at an appropriate level or restored or increased to an appropriate level while pro-inflammatory molecules and/or cell adhesion molecules are reduced. The serum levels of the phenolic compounds may be between about 10 μM to 100 mM. Preferably, serum levels of the phenolic compounds may be between about 50 μM to 1 mM. More preferably, serum levels of the phenolic compounds may be 75 μM to 500 μM.

High intake of whole grain food is associated with reduced risk of colon cancer, but the mechanism underlying this protection has yet to be elucidated. Avenanthramides (Avns) are a unique polyphenol mainly present in oats. Chronic inflammation has been suggested to contribute to the increased risk of colon cancer. The effect of Avns of oat extract and CH₃-Avn-C on colon cancer cell proliferation, COX2 expression and the signaling pathway leading to its expression is shown in Example 15. Both Avns of oat extract and CH₃-Avn-C significantly inhibited cell proliferation of Caco2, HCT116, LS-174, and HT29 colon cancer cells. The order of the sensitivity to Avns oat extract and CH₃-Avn-C according to their IC₅₀ was: HCT116>Caco2>LS-174>HT29. In contrast Avns had no effect on the confluence-induced differentiated Caco2 cells, which represents the normal colonic epithelial cells. COX2 protein expression induced by TNF-α stimulation in HT29 cells was inhibited by either Avns oat extract or CH₃-Avn-C, but they had no effect on COX1 expression. COX2 protein expression can be inhibited through administration of the phenolic compounds of the present invention by 20%, 40%, 60%, 70%, 80%, 90%, or greater than 95% compared to normal levels. The prostaglandin E2 (PGE2) level in HT29 cell culture medium was decreased by pretreatment with either Avns oat extract or CH₃-Avn-C. They also suppressed the phosphorylation of the p65 subunit of NFκB in TNF-α stimulated HT29 cells. CH₃-Avn-C has a higher capacity than Avns oat extract of all the effects stated above. Phosphorylation of the p65 subunit of NFκB can be inhibited by approximately 20%, 40%, 60%, 70%, 80%, 90%, or greater than 95% compared to normal levels. Example 15 shows that oat Avns play an important role in regulating inflammation of colon cancer and inhibiting colon cancer proliferation. The anti-proliferative effect of Avns is independent of its anti-inflammatory effect.

Inflammation enhances tumor development and inflammatory bowel disease (IBD) is a risk factor for colon carcinogenesis. Several non-steroidal anti-inflammatory drugs and food component such as genistein, curcumin suppresses colonic inflammation as well as reduce colon cancer risk. In addition, patients with ulcerative colitis and Crohn's disease are at increased risk for developing colon cancer. To date, no known genetic basis has been identified to explain colorectal cancer predisposition in these diseases (Itzkowitz, S. H. and X. Yio, Am J Physiol Gastrointest Liver Physiol, 2004. 287(1): p. G7-17). Chronic inflammation of gastrointestinal tract is believed to be main culprit for initiation of colon cancer (Prescott, S. M. and R. L. White, Cell, 1996. 87(5): p. 783-6). Cellular signaling activated by proinflammatory conditions and oxidative stress can lead to the initiation and progression of cancer by inducing DNA damage and epigenetic changes, increased sensitivity to growth factors and abnormalities in the expression and activity of transcription factors that control cell cycle progression and survival. Consequently, both antioxidants and anti-inflammatory agents markedly inhibit the initiation and development of cancer including colon cancer in animal models as well as in humans. For example, dietary antioxidants such as vitamin E, selenium, β-carotene, and other phytochemicals neutralize reactive oxygen species and act as cancer preventive agents.

Intake of non-steroidal anti-inflammatory drugs (NSAIDs) such as aspirin has been suggested to reduce the risk of colorectal cancer. Dietary polyphenols such as resveratrol, curcumin, and EGCG inhibited the growth of several immortalized human colorectal cancer cell lines and their antitumorigenic effects have been linked in part to inhibition of cyclooxygenase-2 (COX-2) expression. The protective effects of NSAIDs are also thought to be mediated through an inhibition of COX activity which catalyzed the formation for prostaglandins from arachidonic acid. Up to now, two isofroms of COX, designated as COX-1 and COX-2, have been identified and characterized in animal models and humans. COX-2, but not COX-1, is overexpressed in 80-85% of human adenocarcinomas and colonic tumors and in 80-85% of ApcMin/+ mouse adenomas.

The methods of the present invention could also be used to treat, reduce, or prevent colon cancer. Avenanthramides (Avns) are unique phenolic compounds present mainly in oat and oatmeal and exhibit antioxidant activity. Prior to this invention, no information was available on the effects of Avns on the colon cancer cell proliferation and inflammatory signaling pathway involved in cancer initiation and progression. Example 15 shows experiments that evaluate the efficacy of Avns extracted from oats and that of synthetically prepared (CH₃-Avn-C) on proliferation of colonic cancer cell lines and the expression and signaling pathways involved on COX2 expression which plays a central role in the inflammation and colon carcinogenesis.

In some embodiments, the phenolic compounds of the present invention can be combined with other agents to produce an enhanced or synergistic effect resulting in a reduction or decreased risk of colorectal cancer. Besides dietary fiber, other components found in whole grains such as vitamins, minerals and polyphenols have been indicated to contribute to their protection from colon cancer. Exemplary agents that can be combined with the phenolic compounds of the present invention include, but are not limited to, vitamin E, B vitamins, zinc, selenium, copper, iron, manganese, magnesium, β-carotene, and other phytochemicals, NSAIDs, and polyphenols (such as resveratrol, curcumin, and EGCG). The NSAID can be selected from the group consisting of ibuprofen, sulindac, ketoporfen, fenoprofen, flurbiprofen, naproxen, tiaprofenic acid, suprofen, etodolac, carprofen, ketrolac, piprofen, indoprofen, celecoxib, rofecoxib, mobicox, and benoxaprofen. Only part of the ingested polyphenols is absorbed, suggesting that much of the compounds may have a local effect on the epithelial cells of the colon.

Example 15 explored the effect of phenolic compounds on COX2 expression in colon cancer cells. COX2 is an important inflammatory mediator which is found to be upregulated in more than 80% of colon cancers. A commonly used model to study the effects on COX2 is the human colonic epithelial carcinoma cell line HT29 (Glinghammar, B. and J. Rafter, Gastroenterology, 2001. 120(2): p. 401-10; Yamazaki, R., et al., FEBS Lett, 2002. 531(2): p. 278-84). There is a basal low level expression of COX2 in these cells and TNF-α stimulation can upregulate its expression. TNF-α is a cytokine naturally present in the colon. It upregulates COX2 via activation of nuclear factor (NF)kB transcription factor. Example 15 shows that Avns significantly inhibited TNF-α induced COX2 expression in a dose-dependently manner. Inhibitors of COX2 activity can be effective as anti-inflammatory agents and also in the prevention and treatment of colon caner. Prostaglandins (PGs) are mediators of inflammation, and chronic inflammation predisposes to carcinogenesis. Avns can be used as a COX2 specific inhibitor which are be devoid for the side effects associated with NSAIDs.

Nuclear factor kappa B (NF-kB) regulates transcription of a variety of genes which control inflammation, survival, and proliferation. It has been reported that NF-kB activity can be inhibited by nitric oxide (NO). As discussed above, phenolic compounds, such as Avns, increase NO production. Therefore, NO might play a role in mediating the inhibitory effect of Anvs on NF-kB activation. NFkB signaling pathways provide attractive targets for new chemopreventive and chemotherapeutic approaches. As shown in Example 15, phenolic compounds of the present invention can be used as a natural potent anti-tumor drug.

As shown below, Avns significantly inhibited the proliferation of growing phase of Caco2 cells but have little effect on the differentiated CaCo2 cells, which exhibit normal characteristics of colonic epithelial cell. CH₃-Avn-C has a stronger inhibitory effect (IC₅₀ of 30 μM) compared with Avns oat. The data showed that HCT cells, which is COX2 negative, is more sensitive to Avns than the other colon cancer cell lines, which are COX2 positive. These data indicate that the growth inhibitory effects of Avns is independent on COX2 pathway. Therefore, the mechanisms of Avns for inhibiting colon cancer cell proliferation is different from that of other polyphenols, such as curcium or EGCG, whose inhibitory effects are related to COX expression level.

III. Phenolic Compound Compositions

One or more physiologically acceptable phenolic composition can be formulated in a form suitable for topical application. This could be useful for treating skin inflammation due to autoimmune diseases such as Pemphigus folliaceus, pemphigus vulgaris, psoriasis, sarcoidosis, scleroderma, Sjögren's syndrome, rheumatoid arthritis, system lupus erythematosus (SLE), or scleroderma. For example, as a lotion, aqueous or aqueous-alcoholic gels, vesicle dispersions or as simple or complex emulsions (O/W, W/O, O/W/O or W/O/W emulsions), liquid, semi-liquid or solid consistency, such as milks, creams, gels, cream-gels, pastes and sticks, and can optionally be packaged as an aerosol and can be in the form of mousses or sprays. The composition can also be in a sunscreen. These compositions are prepared according to the usual methods. The composition can be packaged in a suitable container to suit its viscosity and intended use by the consumer. For example, a lotion or cream can be packaged in a bottle or a roll-ball applicator, or a propellant-driven aerosol device or a container fitted with a pump suitable for finger operation. When the composition is a cream, it can simply be stored in a non-deformable bottle or squeeze container, such as a tube or a lidded jar. The composition may also be included in capsules such as those described in U.S. Pat. No. 5,063,507.

One or more physiologically acceptable phenolic compound can be administered as compositions by various known methods, such as by injection (subcutaneous, intravenous, etc.), oral administration, inhalation, transdermal application, or rectal administration. Depending on the route of administration, the composition may be coated with a material to protect the compound from the action of acids and other natural conditions which may inactivate the compound. The composition can further include both the avenanthramide compound and another agent, such as a cholesterol-lowering agent.

To administer the composition by other than parenteral administration, it may be necessary to coat the composition with, or co-administer the composition with, a material to prevent its inactivation. For example, the composition may be administered to a subject in an appropriate diluent or in an appropriate carrier such as liposomes. Pharmaceutically acceptable diluents include saline and aqueous buffer solutions. Liposomes include water-in-oil-in-water CGF emulsions as well as conventional liposomes (Strejan et al., J. Neuroimmunol. 7:27 (1984)).

The composition containing at least one phenolic compound of the present invention may also be administered parenterally or intraperitoneally. In some embodiments, the at least one avenanthramide comprises Av-C. In a preferred embodiment, the at least one avenanthramide comprises the methyl ester form of the compound. Dispersions can also be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene gloycol, and the like), suitable mixtures thereof, and vegetable oils. The proper fluidity can be maintained, for example, by the use of a coating such as licithin, by the maintenance of the required particle size in the case of dispersion and by the use of surfactants. Prevention of the action of microorganisms can be achieved by various antibacterial and antifungal agents. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the composition containing the antioxidant in the required amount in an appropriate solvent with one or a combination of ingredients enumerated above, as required. Generally, dispersions are prepared by incorporating the composition into a sterile vehicle which contains a basic dispersion medium and the required other ingredients from those enumerated above.

When the composition containing the antioxidant is suitably protected, as described above, the composition may be orally administered, for example, with an inert diluent or an assimilable edible carrier. The composition and other ingredients may also be enclosed in a hard or soft shell gelatin capsule, compressed into tablets, or incorporated directly into the subject's diet. For oral therapeutic administration, the composition may be incorporated with excipients and used in the form of ingestible tablets, buccal tablets, troches, capsules, elixirs, suspensions, syrups, wafers, and the like. The percentage of the compositions and preparations may, of course, be varied. The amount of active compound in such therapeutically useful compositions is such that a suitable dosage will be obtained.

The tablets, troches, pills, capsules and the like may also contain a binder, an excipient, a lubricant, or a sweetening agent. Various other materials may be present as coatings or to otherwise modify the physical form of the dosage unit. For instance, tablets, pills, or capsules may be coated with shellac, sugar or both. Of course, any material used in preparing any dosage unit form should be pharmaceutically pure and substantially non-toxic in the amounts employed. As used herein “pharmaceutically acceptable carrier” includes any solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in compositions of the invention is contemplated.

It is especially advantageous to formulate compositions of the invention in dosage unit form for ease of administration and uniformity of dosage. Dosage unit form as used herein refers to physically discrete units suited as unitary dosages for the subjects to be treated. Each dosage contains a predetermined quantity of active compound calculated to produce the desired therapeutic effect in association with the required pharmaceutical carrier. The specification for the novel dosage unit forms of the invention is dependent on the unique characteristics of the composition containing the antioxidant and the particular therapeutic effect to be achieved. Dosages are determined by reference to the usual dose and manner of administration of the ingredients.

IV. Uses

Many disorders or diseases arise due to oxidative stress, the presence of free radicals, and altered immune responses that can lead to inflammatory diseases and cardiovascular diseases. The methods of the present invention can be used to help treat, prevent, or reduce disorders associated with excess free radicals, inflammatory molecules, or adhesion molecules. Examples of such disorders, include, but are not limited to segmental progeria disorders, Down's syndrome; heart and cardiovascular diseases such as atherosclerosis, adriamycin cardiotoxicity, asthma, alcohol cardiomyopathy; cardiovascular disorders such as post-angioplasty restenosis, coronary artery disease, and angina; gastrointestinal tract disorders such as inflammatory & immune injury, diabetes, pancreatitis, halogenated hydrocarbon liver injury; eye disorders such as cataractogenesis, degenerative retinal damage, macular degeneration; kidney disorders such as autoimmune nephrotic syndromes and heavy metal nephrotoxicity; skin disorders such as solar radiation, dermatitis, thermal injury, porphyria: nervous system disorders such as hyperbaric oxygen, Parkinson's disease, neuronal ceroid lipofuscinoses, Alzheimer's disease, muscular dystrophy and multiple sclerosis; lung disorders such as lung cancer, oxidant pollutants (O₃, NO₂), emphysema, bronchopulmonary dysphasia, asbestos carcinogenicity; red blood cell disorder such as malaria Sickle cell anemia, Fanconi's anemia and hemolytic anemia of prematurity; iron overload disorders such as idiopathic hemochromatosis, dietary iron overload and thalassemia; inflammatory-immune injury, for example, glomerulonephritis, autoimmune diseases, rheumatoid arthritis; ischemia reflow states disorders such as stroke and myocardial infarction; liver disorder such as alcohol-induced pathology and alcohol-induced iron overload injury; and other oxidative stress disorders such as AIDS, radiation-induced injuries (accidental and radiotherapy), general low-grade inflammatory disorders, organ transplantation, osteoarthritis, inflamed rheumatoid joints and arrhythmias.

In addition, many disorders or diseases can be attributed to a lack of or need for nitric oxide (NO) including prohibiting, preventing, restraining, and slowing, stopping, or reversing progression, severity or a resultant symptom or effect of the physiological condition. The methods of the present invention can be used to help treat, prevent, or reduce disorders due to a lack of or need for nitric oxide (NO). Such physiological conditions include, pathological platelet aggregation, pathological vasoconstriction, vascular effects of diabetes, stroke, atherosclerosis, hypertension, abnormal vasospasm, and restenosis after angioplasty.

(i) Prophylaxis and/or Treatment

The methods of the invention could be used to prevent or delay onset of disease. For example, people at high risk for cardiovascular disorders could use the purified avenanthramides to reduce their risk. The effect of avenanthramides on smooth muscle proliferation and nitric oxide production of endothelial cells and smooth muscle cells, which play a pivotal role in the initiation and progression of atherosclerosis, was elucidated through the use of the present invention as shown in the Examples.

In one aspect, the methods of the present invention may be used to treat or reduce atherosclerosis. Atherosclerosis is one of the leading cause of morbidity and mortality in western society and is the underlying cause of cardiovascular disease (heart disease and stroke). Overwhelming evidence indicates that inflammatory process plays an important role in the pathogenesis of the disease through the interaction between vascular endothelium and immune cells, in which a variety of mediators including pro-inflammatory cytokines, chemokines and adhesion molecules participate in the initiation and progression of atherosclerosis (Stemme, S. et al. Ann Med. 26: 141-6 (1994)). The effect of oat extract on immune and endothelial cells interactions, which is considered to play a pivotal role in the initiation and progression of atherosclerosis, was elucidated through the use of the present invention as shown in the Examples. Phenolic compounds of the present invention can be used to inhibit smooth muscle cell proliferation. In a preferred embodiment, alkyl ester forms of the phenolic compounds of the present invention are used to inhibit smooth muscle cell proliferation.

Proliferation of SMC in the injured arterial wall during the development of the atherosclerosis significantly contributes to the intimal thickening and restenosis following angioplasty and stent application. The in vitro cell culture results, shown in the Examples, demonstrate for the first time that synthetically prepared Avn-c, one of the major unique polyphenols found in oats, inhibits serum-induced SMC through arresting cell division at G1 phase (see Example 9). This inhibitory effect of Avn is mediated by several key growth-regulatory proteins that are known to play important roles in cell division, including pRb, cyclin D1, P21 and p53.

Mitogen-induced vascular SMC proliferation is inhibited by many agents such as retinoids, sodium salicylate, and doxazocin. Most of these agents inhibit pRb phsophorylation. pRB is the major negative regulator of cell division and exerts most of its effect in the first two-thirds of the G1 phase of the cell cycle. Hypophosphorylated pRB has been shown to bind to the transcription factor E2F-1 resulting in an inhibition of E2F-dependent transcriptions including the genes for thymidine kinase dihydrofolate reductase, which are important for the cell cycle progression. In contrast, phosphorylation of pRB leads to the inactivation of its growth inhibitory effects and causes cell entry into the S phase. The Examples clearly show that Avn blocks the serum-induced phosphorylation of pRB in a dose-dependent manner. Since phosphorylation of pRB occurs at the late G1 phase, the inhibition of pRB phosphorylation by Avn explains the cell cycle arrest that observed at G1 phase.

A key molecule that regulates the phosphorylation of pRB is cyclin D1, which is required for cell cycle progression in G1 phase. The expression of cyclin D1 increases in response to mitogenic stimulation and rapidly declines when growth factors or mitogens are withdrawn. Example 10 demonstrates that FBS increased the cyclineD1 levels in quiescent A10 cell and Avn dose dependently suppressed the expression of this cell cycle regulatory protein. Transcriptional regulation of cyclin D1 is a key event in G1 progression. Activation of Ras increases the cyclin D promoter activity and the MAP kinase is thought to be involved in this Ras-mediated transcription regulation.

P21cip1 is a universal inhibitor of cyclin dependent kinase (CDK), which phosphorylates pRb. Overexpression of p21 by means of adenoviral gene delivery effectively prevented neointiaml formation. The levels of P21cip1 in quiescent vascular SMC were high and decreased after serum stimulation (See, Example 12). However, the expression of this cell cycle regulatory protein increases with Avn in a dose-dependent manner. It is plausible that the increase in p21cip1 expression with Avn treatment might have lead to the inhibition of CDK activity, which in turn resulted in the inhibition of pRb phosphorylation and cell cycle arrest.

The control of proliferation of vascular SMC can be regulated by p53. P53 is a key tumor suppressor protein and transcription factor of p21cip1. It has been shown that transfection of the bovine SMC with exogenous p53 caused cell cycle arrest at G1 phase. However, induction of cell cycle arrest is mainly mediated by upregulating p21cip 1. Given that Avn treatment increased p21cip1 expression but has no effect on p27kip1 level, the changes in p53 levels with Avn might be responsible for p27kip1 and cell cycle arrest in G1 phase. Avn increased p53 protein level up to 3-fold (See, Example 11). Post-translational modifications of p53 is important for stabilization and activation this cell cycle regulatory protein. Example 13 demonstrates that Avn treatment of A10 cells increased the half-life of p53 from about 20 min to more than 1 h. This increased stability with Avn treatment might have contributed to the higher observed levels of p53 protein and up-regulation of p21cip1 expression. This invention demonstrates that phenolic compounds of the present invention can inhibit SMC proliferation at the G1 phase by up-regulating the p53-p21cip1 pathway and inhibiting pRB phosphorylation.

In another aspect, the present invention can be used as a vasodilator due to its increase of the nitric oxide production. Nitric oxide (NO) in the blood exercises various biochemical functions. Nitric oxide serves as an important messenger molecule in the brain and other parts of the body, governing diverse biological functions. In blood vessels, the principal endothelium-derived relaxing factor (EDRF) is believed to be nitric oxide, which stimulates vasodilation. Nitric oxide also inhibits platelet aggregation and is partially responsible for the cytotoxic actions of macrophages. In the brain, nitric oxide mediates the actions of the excitatory neurotransmitter glutamate in stimulating cyclic GMP concentrations. Immunohistochemical studies have localized nitric oxide synthase (NOS) to particular neuronal populations in the brain and periphery. Inhibitors of nitric oxide synthase block physiological relaxation of the intestine induced by neuronal stimulation, indicating that nitric oxide has the properties of a neurotransmitter. In this regard, nitric oxide appears to be a novel type of neuronal messenger, in that, unlike conventional neurotransmitters, nitric oxide is not stored in synaptic vesicles and does not act on typical receptor proteins of synaptic membranes. One function of nitric oxide may be to protect neurons from ischemic and neurotoxic insults. See, Bredt et al., “Cloned and Expressed Nitric Oxide Synthase Structurally Resembles Cytochrome P-450 Reductase,” Nature, Vol. 351, June, 1991, pages 714-718.

In another aspect, the methods of the invention could be used to prevent or delay onset of cardiovascular disease. For example, the reduction of pro-inflammatory cytokines and adhesion molecules has been associated with decreased risk for cardiovascular disease. Both the heart and the blood vessels are sensitive to the effects of pro-inflammatory cytokines as well as vasoactive substances. Nitric oxide is synthesized by inducible nitric oxide synthase (iNOS) in the vascular endothelium and smooth muscle in response to pro-inflammatory cytokines. People at high risk for such disorders could use the purified avenanthramides to reduce their risk.

In another embodiment, the methods of the present invention can be used to enhance resistance to certain disease states. The oxidant/antioxidant balance plays an important role in the pathogenesis of atherosclerosis. The synthesis of pro-inflammatory cytokines, chemokines, and eicosanoids are regulated by redox status through gene activation and post-transcription regulation. Antioxidants can reduce reactive oxygen species and modulate cytokine, chemokine, and eicosanoid production, which may contribute to the potential beneficial effects of antioxidants on reducing the risk of cardiovascular disease.

The methods of the invention can be used to treat immune disorders, such as inflammatory disorders or autoimmune disorders, caused by modified immune responses. In one embodiment, the methods and compositions of the present invention can be used to treat atherosclerosis. In another embodiment, the methods and compositions of the present invention can be used to modulate tumor growth. Significant amounts of IL-8, MCP-1 and IL-6 are produced by endothelial cells, smooth muscle cells and macrophages when they become activated with cytokines or mitogens. IL-8 is a potent chemoattractant to neutrophils, lymphocytes, and basophils and may contribute to recruitment of inflammatory cells in atherosclerotic plaque. IL-8 is produced by the adhesive interaction of endothelial cells and monocytes during the transmigration of monocytes through monolayers. The lymphocytes recruitment to the site of activation in turn increases reactive oxygen species and decreases antioxidant defense. In addition, IL-8 is reported to be chemoattractant for human aortic smooth muscle cell. Further, IL-8 is a potent angiogenic factor which contributes to the growth of atherosclerotic lesions (Simonini et al Circulation. 101: 1519-1526 (2000)) and tumor growth. MCP-1 is a powerful monocyte chemoattractant, both in vivo and in vitro, and has been shown to be expressed by endothelial cells in early atherosclerotic lesions and involved in monocyte/macrophage recruitment to early lesions. Cushing et al. (Cushing et al. Proc Natl Acad Sci USA. 87: 5134-5138 (1990)) reported that endothelial cells and SMCs of vascular wall upregulated production of MCP-1 when exposed to modified LDL. MCP-1 was also found in macrophage-rich areas of atherosclerotic lesions, but was absent in nonlesional areas. In addition, COX-2 expression and PGE2 is also involved in promoting angiogenesis. In one embodiment, the methods and compositions of the present invention reduce COX-2 expression and PGE2. Thus, the invention can be used to decrease angiogenesis, which can be used in the treatment of tumor growth.

In another embodiment, the methods of the invention can be used to prevent the formation of fatty streaks or atherosclerotic plaques. In addition to monocytes, T lymphocytes are important components in the formation of atherosclerotic plaques. IL-6 has been shown to increase adhesion of circulating lymphocytes, particularly CD4+ to HLUVEC and enhanced the expression of ICAM-1, VCAM-1 and E-selectin on endothelial cells. IL-6 is also one of the principal growth-regulatory molecules responsible for the migration and proliferation of SMC. Therefore, this cytokine, together with other chemokines, play a pivotal role in the formation of fatty streaks. Examples 4 and 5 show that incubating HAEC with oat extract reduces the expression of adhesion molecules and of IL-6, respectively.

The methods of the invention may be used to modulate blood lipids. In another embodiment, the methods of the invention may help to reduce CVD risk. It is important to note that Tranilast, [N-(3′4′-dimethoxycinnamoyl)-anthranilic acid], a drug capable of preventing angiographic restenosis after percutaneous transluminal coronary revascularization (Rosanio et al. Thromb Haemost. 82 Suppl 1: 164-170 (1999)), shares a very similar structure with avenanthramide. Tranilast inhibition of restenosis is through the inhibition of vascular smooth muscle cells proliferation and migration (Miyazawa et al. Atherosclerosis. 118: 213-21 (1995); Takahashi et al. Circ Res. 84: 543-50 (1999)) and via inhibition of production of various cytokines from inflammatory cells (Chikaraishi et al. Eur J. Pharmacol, 427: 151-158 (2001)). Oat is unique in the sense of its complement of phenolic compounds, which have powerful antioxidant function associated with the capability of the oxygenated aromatic moiety to scavenge free radicals (Collins, F. W., Quaker Oats Phenolic Antioxidant Study. First Semi-Annual Progress Report, 2000). Avenanthramide are the major phenolic antioxidant identified in oats. Three major Avenanthramides A, B and C are the three major avenanthramides composed of more than 75% of the total antioxidant phenolics present in the avenanthramide fraction, which are closely correlated with antioxidant activity (Emmons et al. J. Agric Food Chem. 47: 4894-8 (1999)).

In another embodiment, the present invention may be effective in the reduction, treatment, or prophylaxis of inflammation and pain, including but not limited to chronic gastritis, arthralgia, benign prostate hyperplasia, chronic and recurrent cystitis, cervical disc, degenerative joint arthritis, rheumatoid arthritis, tennis elbow, osteoportoric pain, migraine, diabetic neuropathy pain and flank pain. Oat extract exhibits high capacity to inhibit adhesive interaction between endothelial cells through inhibition of adhesion molecule expression and to inhibit cytokines and chemokines that are important in the recruitment of immune cells to the site of inflammation.

In yet another embodiment, the methods of the invention can be used to decrease tissue injury and the inflammatory response. Polymorphonuclear leucocytes are one of the main cellular mediators of tissue injury. They accumulate in tissues in response to endotoxin and pro-inflammatory cytokines mediated through IL-8, a powerful chemoattractant and activator of polymorphonuclear leucocytes. Tissue injury occurs due to degranulation of the leucocytes producing proteases (i.e., elastase and matrix metalloproteinases) and the production of reactive oxygen species (ROS). Activated neutrophils produce large amounts of ROS from membrane bound NADPH oxidase which produces the oxygen free radical superoxide and hydroxyl radical. These have been implicated in tissue injury but are also part of the microbial cytotoxic system of the neutrophil. In another aspect of the invention, the alcoholic oat extract and/or avenanthramide can be incorporated into a topical treatment for inflammation that arises due to the allergenic or immune response.

In another embodiment, the invention can be used to treat or reduce respiratory dysfunction. Pulmonary dysfunction can be manifested as tachypnea, hypoxemia and respiratory alkalosis. When severe it may progress to acute lung injury (ALI) and acute respiratory distress syndrome (ARTS). The primary pathological process is pulmonary capillary endothelial dysfunction resulting in interstitial and alveolar edema of protein and phagocytic immune cell rich exudative fluid. Endothelial permeability is increased in response to pro-inflammatory cytokines with progression to alveolar denudation and basement membrane destruction. Neutrophils are sequestrated into the lung in response to IL-8. Concentrations of IL-8 in lung broncheoalveolar lavage fluid in patients with ARDS has been shown to correlate with mortality. In one embodiment, avenanthramides and/or an oat extract composition can be aerosolized and inhaled directly to reduce pro-inflammatory cytokines, such as IL-8.

In another embodiment, the methods of the invention can be used to treat renal dysfunction. Several mechanisms have been proposed for the pathogenesis of acute renal failure. In normal states, the kidney maintains renal blood flow and glomerular filtration through autoregulation dependent on the tone of the afferent and efferent arterioles. The cytokine-induced systemic vasodilatation and relative hypovolemia in diseased states are responsible for renal hypoperfusion. The kidney produces intrinsic vasoconstrictors in response to cytokines and the renin-angiotensin-aldosterone system. In common with other tissues, the kidney is susceptible to leukocyte mediated tissue injury with neutrophil aggregation in response to chemokines and production of proteases and ROSs. In one embodiment, the oat extract composition and/or phenolic compounds can be coupled to a delivery vehicle such that the controlled release of the active ingredient is achieved.

Several inflammatory cytokines including interleukin IL-1, tumor necrosis factor (TNF), and interferon produced by activated monocytes and macrophages may stimulate the endothelium to up-regulate genes encoding for chemokines, other cytokines as well as adhesion molecules which mediate attraction and adhesion of immune cells to the endothelium. The upregulated expression of numerous cell surface adhesion molecules is a critical process for the binding of normally non-thrombogenic circulating leukocytes such as the monocytes to the arterial endothelial surface and is one of the earliest detectable events in atherosclerosis. Increased expression of adhesion molecules such as intracellular adhesion molecule-1 (ICAM-1), vascular cell adhesion molecule-1 (VCAM-1), and endothelial leukocyte adhesion molecule-1 (E-selectin) mediate the transmigration of leukocytes to subendothelium leading to the formation of atheromatous plaques. Cytokines are actively involved in leukocyte recruitment, activation and migration. Both activated immune cells and endothelial cells produce a variety of cytokines during the atherogenesis. IL-8 is chemoattractant to T cells and neutrophils, increases proliferation and migration of vascular smooth muscle cells (SMC), and is an angiogenic factor. Monocyte chemoattractant protein-1 (MCP-1), in addition of chemoattractant activity on monocyte and basophils, is a strong cytokine to convert monocyte rolling to firm adhesion on the endothelium expressing E-selectin under flowing conditions. IL-6 is another pro-inflammatory cytokines that can act as a mitogenic stimuli and be responsible for the migration as well as proliferation of SMC.

Dietary factors are known to play significant etiologic roles in the development of atherosclerosis. Diet affects the development of atherosclerosis not only through modulation of lipoprotein metabolism, but also by influencing the inflammatory processes associated with the development of this disease. Production of chemokines and adhesion molecules by endothelial cells has been shown to be regulated by redox sensitive signal transduction and thus may be subjected to modulation by oxidants and antioxidants.

Previous reports indicate that dietary vitamin E, lycopene and polyphenolics with antioxidant activity inhibit adhesion of immune cells to endothelial cells through modulation of cytokines production, adhesion molecules expressions, and chemokines release in vivo and in vitro. Consumption of oat is associated with reduced risk of coronary heart disease and has been shown recently to improve endothelial dysfunction (Katz, D. L. et al. Prev Med. 33: 476-84 (2001)). Like all monocot cereals, oats (Avena sativa L.) contain a number of phytochemicals containing a phenolic moiety with free-radical scavenging capability and thus exhibiting antioxidant properties in vitro.

The active interaction of the endothelium with cells of the immune system is widely acknowledged, particularly as it relates to inflammatory processes and foam cell formation in the development of atherosclerosis. Stimulation of either cell type, viz., human arterial endothelial cells (HAEC) or monocytes, results in expression of numerous adhesion molecules and/or counter-ligands, as well as induces further secretion of other pro-inflammatory factors, including cytokines and chemokines. As this process continues, the monocytes, which typically roll, are triggered, activated to bind strongly, and stick to the endothelium, eventually infiltrating where plaque formation is initiated. Stimulation of HAEC and/or monocytes of human origin represents an effective model for assessing the early events of vascular modification and atherogenesis. Enrichment of HAEC or monocytes with antioxidants, (i.e., vitamin E, probucol, N-acetylcysteine, and pyrrolidine dithiocarbamate) decreases adhesion and interaction of EC with immune/inflammatory cells.

The methods of the invention can be used to reduce the production or secretion of cell adhesion molecules. In a preferred embodiment, the methods of the invention can be used to reduce fatty streaks that may lead to diseased states. Cell adhesion molecules (CAM) play an important role in the development of the early fatty streaks and fibrous atherosclerotic plaques. VCAM-1 expressed by endothelial cells, through binding to VLA-4 integrin mediates the adhesion of leukocytes to activated endothelium. ICAM-1 through binding to LFA-1 and MAC-1 integrins is involved in the adhesion of leukocytes and neutrophils to the endothelium during activation, flattening, and extravasation. Increased expression of ICAM-1 in the atherosclerotic plaques has been demonstrated by the presence of an increased immunoreactivity to ICAM-1 antibody. The increased E-selectin expression in endothelial cells surface induced by cytokines, bacterial toxins, and oxidants also mediate immune/endothelial cell adhesions. While ICAM-1 is constitutively expressed by the normal unstimulated endothelial cells, the expression of VCAM-1 and E-selectin is negligible.

The methods of the invention can be used to decrease cell expression of adhesion molecules, production of chemokines and pro-inflammatory of cytokines. Endothelial cells do not normally express elevated levels of adhesion molecules and thus, do not normally support excessive attachment to leukocytes. However, such an interaction is stimulated by exposure to a number of stimuli, including LDL, oxidized LDL, bacterial lipopolysaccharide, and inflammatory cytokines such as IL-1β, which induces phenotypic changes. The cytokine- and/or chemokine-stimulated adhesion of monocytes to endothelium of bovine, porcine, and human origin has been routinely used as a model to investigate interactions between leukocytes and the endothelium. Pretreatment of HAEC with oat extracts dose-dependently reduced IL-1β stimulated HAEC expression of adhesion molecules, productions of chemokines and pro-inflammatory of cytokines and their adherence to U937 cells as shown in Examples 3-5. The effect of enrichment of human aortic endothelial cells (HAECs) on their adherence to monocyte and production of inflammatory cytokines and chemokines is shown in Examples 3, 4, and 5

(ii) Prevention

In one aspect, the methods of the invention can be used to reduce the risk, prevent the disorder or delay onset of cardiovascular disorders. The method of the invention can be used to reduce the risk, prevent or delay onset of heart disease. In one embodiment, the compounds of the present invention can be incorporated into polymers, such as those used to make cardiovascular stents, or used as a coating on stents to prevent proliferation of SMC and prevent restenosis after angioplasty. The compounds of the present invention can, for example, be combined with and/or impregnated into polymers (e.g., biodegradable polymers, slow release polymers, and/or controllable or inducible-release polymers) such that the compound can be delivered to the target site over time. The polymer can be impregnated with one or more compound of the present invention such that release can be controlled and directed to the target area (e.g., vascular cells). In addition, the stents can comprise one of more compounds of the present invention combined with other compounds (e.g., antioxidants, such as vitamin E, and other phenolic compositions) to provide synergist effects and/or with other drugs (e.g., antibiotics, growth factors, cholesterol reducing agents, such as statins, anti-neoplastics, immunosupressives, migration inhibitors, and enhanced healing factors) to reduce the risk of restenosis and/or intimal hyperplasia.

In another aspect, the methods of the invention can be used to reduce the risk, prevent the disorder or delay onset of oxidative stress disorders. The method of the invention may have uses in endurance exercise training by protecting against oxyradical increase. The method of the invention could have applications in vascular biology such as reducing oxidative stress or inhibit foam cell production. In another embodiment, the method of the invention could be used to improve vision as the avenanthramides could be transported to the eye lens and macular and reduce cataract formation. In yet another embodiment, the method of the invention could have uses in neuroscience by affecting signal transduction pathways.

The method of the invention can be used to reduce the risk, prevent or delay onset of inflammatory disorders and/or heart disease. Leukocyte adhesion to the endothelium is a multi-stage process occurring early in the pathogenesis of atherosclerosis and inflammation. In a preferred embodiment, the invention can be used to prevent onset of the disease. In response to infection and tissue injury, endothelial cells become activated and express molecules through the generation of vasoactive compounds, lipid-based activators, chemokines, and specific cell surface adhesion molecules. Leukocyte adhesion to the endothelium is facilitated by the presence of leukocyte adhesion molecules or receptors. Intercellular adhesion molecule-1 (ICAM-1) is expressed by resting endothelial cells. Conversely, endothelial-leukocyte adhesion molecule-1 (ELAM-1) and vascular cell adhesion molecule-1 (VCAM-1) are normally absent in resting endothelial cells, but their expression can be induced by various plasma components, including cytokines such as interleukin-1β. LDL cholesterol, cytokines, primarily IL-1β and tumor necrosis factor (TNF), nitric oxide, and oxidized lipoproteins modulate the adhesiveness of the endothelium via stimulating adhesion molecule expression. The similarities between inflammation and atherogenesis suggest a role for ROS in these processes. In one embodiment, the methods of the invention can be employed to increase cellular accumulation of antioxidant compounds in the endothelium which can reduce the expression of adhesion molecules by quenching free radical production. In fact, increased expression of adhesion molecules, such as VCAM, and marked enhancement of monocyte adhesion after stimulation of human umbilical vein endothelial cells with IL-1β were both significantly inhibited after preincubation of monolayers with the antioxidants.

In one embodiment, the methods of the present invention can also be used in food supplements and nutraceutical formulations. A nutraceutical refers to formulations of natural or naturally-derived agents that can impart medical and/or health benefits. Thus, oat extract and/or phenolic compositions described in this invention can be incorporated into supplements or formulated as nutraceutical supplements themselves. In addition, foodstuff can be enhanced with phenolic compositions, such that the enhanced foodstuff can delay onset of inflammatory disorders and/or heart disease. In another embodiment, a composition comprising of an alcoholic extract of oats can be utilized as a nutraceutical supplement. In a preferred embodiment, the supplement would comprise substantially purified phenolic compounds.

This invention is further illustrated by the following examples which should not be construed as limiting. The contents of all references, patents and published patent applications cited throughout this application, are incorporated herein by reference.

### EXAMPLES

The following experiments were performed to establish methods for increasing NO, inhibiting smooth muscle cell proliferation, inhibiting a physiological condition associated with a lack of or need for nitric oxide (NO), and reducing pro-inflammatory molecules and cell adhesion molecules through the administration of the phenolic compounds of the present invention.

### Example 1

### Materials and Methods

(i) Materials

FBS was purchased from GIBCO (Grand Island, N.Y.). Propidium iodide (PI) and DNase-free RNase were obtained from Sigma (Saint Louis, Mich.). Monoclonal antibody against pRB (14001A) was obtained from Pharmingen (San Diego, Calif.). Anti-phosphorylated pRb and anti-p53 antibodies were from Cell Signaling (Beverly, Mass.). p21 (C-19, sc-397) was purchased from Santa Cruz Biotechnology, Inc. (Santa Cruz, Calif.). Anti-CyclinD1 and anti-p27^{kip} antibodies were from Sigma. ECL Western assay kit (RPN 2108) was obtained from Amersham Pharmacia Biotech (Piscataway, N.J.). The BCA protein assay kit was purchased from Pierce Chemical Company (Rockford, Ill.).

(ii) Cell culture

HAEC were purchased from Clonetics Laboratories (San Diego, Calif.) and cultured in MCDB-131 medium (Sigma Chemical, St. Louis, Mo.). Passages 6-8 were used in this study. The culture medium contained 2% fetal bovine serum (FBS) (Gibco, Grand Island, N.Y.), 2 mmol/L L-glutamine (Gibco), 100 U/mL penicillin (Gibco), 100 U/mL streptomycin (Gibco), 1 ug/mL hydrocortisone, 0.01 ug/mL epidermal growth factor (EGF), 0.5 mL bovine brain extract (BBE), 0.5 ug/mL amphotericin B (Sigma). RACE were seeded in the 1% gelatin (Sigma) coated T-75 flasks, 24-well plates and 96-well plates. The medium was changed every other day until the cells grew to confluence. 6-8 passage cells were employed and experiments were conducted in triplicate or quadruplicate. U937 cells (American Type Culture Collection, Rockville, Md.) grew in suspension culture in RPM1-1640 medium (Life Technologies, Grand Island, N.Y.) supplemented with 10% FBS, 2 mmol/L L-glutamine, 100 U/mL penicillin, 100 mg/mL streptomycin.

A10 rat embryonic aortic smooth muscle cell line and its culture media Dulbecco's modified Eagle's medium (DMEM) were purchased from ACTT (Manassas, Va.). A10 cells, a primary rat embryonic aorta cell line, is recognized in the art as a model of human vascular SMC. These cells not only maintain the phenotype of vascular SMC but also have some advantages over primary cultures including morphological and biochemical stability. A10 cells were grown in DMEM containing 10% (v/v) FBS, penicillin (0.075 mg/ml), streptomycin (0.05 mg/ml) and 20 mM HEPES/NaOH (pH 7.4), in a 95% humidified air and 5% CO₂. Stock cells were subcultured every 5 d at 1:10 dilution. The cells were restarted from original frozen stock after every 8-10 passages usage. Cell passages at 10-20 were used for the experiments.

Human Aortic Smooth Muscle Cells (HSMC) are derived from tunica intima and tunica media of normal human, fibrous plaque-free aorta. They are cryopreserved at second passage and can be cultured and propagated at least 16 population doublings. HSMC respond to various factors by proliferating or differentiating (Fager, G. et al, In Vitro Cell. Biol. 25(6):511 (1989); Hoshi, H. et al, In Vitro Cell. Biol. 24(4):309 (1988)). They are a well established cell system for the study of human vascular disorders such as atherosclerosis and stroke (Orekhov, A. N. et al, Lab. Invest. 48:749 (1983)); Jonasson, L. et al, Arteriosclerosis 6(2):131 (1986)).

(iii) Preparation of Oat Avenanthramides

A hulless, “identity preserved” variety of oats can be used. To obtain an enrichment of avenanthramides dry milling can be employed, as described by Gray et al. (Gray et al. “Enrichment of Oat Antioxidant by Dry Milling and Sieving,” J Cereal Sci. 32: 89-98 (2000)).

(iv) Extraction and Preparative Purification of the Avenanthramide Fraction

The pearlings obtained by dry milling can be added to refluxing acidified ethanol and stirred vigorously. The stirring mixture can be removed from the heat, cooled, then centrifuged. The resulting supernatant can be decanted through a filter to give a clear greenish-yellow extract. The insoluble pellet can be re-suspended in the same solvent and the above extraction procedure can be repeated for further purification.

To remove non-phenolic, lipophilic components from this micella, hydrophobic resin can be added and the mixture can be concentrated to dryness in vacuo at 35° C. by rotary evaporation. The dried mixture can be suspended in acidified 30% ethanol and quantitatively transferred to a graduated glass chromatography column containing hydrophobic resin, gravity packed and pre-equilibrated in acidified 30% acidified ethanol. The volume can be eluted with acidified 30% ethanol followed acidified 50% ethanol. The combined eluates can be concentrated to a syrup in vacuo at 35° C. by rotary evaporation. The syrup can be taken up in acidified 30% ethanol and chromatographed on a similar column. The column can be eluted again and the eluate concentrated to a syrup in vacuo at 35° C. by rotary evaporation.

The de-fatted extract can be further purified to remove flavonoids, benzoic and cinnamic acids and polar, non-phenolic components by double chromatography. The absorbed avenanthramides, flavonoids and phenolic acids can be recovered by eluting the column with ethanol and concentrating the eluate to dryness in vacuo at 35° C. by rotary evaporation. The dry residue can be taken up in acidified 50% ethanol and loaded onto a second size exclusion column. The column can be eluted with acidified 50% ethanol to remove the flavonoids and phenolic acids and the absorbed avenanthramides recovered with 95% ethanol. This purified “avenanthramide fraction” can be freeze-dried to a deep orange powder after concentration to a small volume in vacuo at 35° C. by rotary evaporation and dilution with water. Various methods for obtaining such extracts are known in the art. Non-limiting examples include Dimberg Hall et al. (Cereal Chemistry 70: 637-641 (1993)), Dimberg et al. (J. Cereal Science 24: 263-272 (1996)), Xing et al. (JAOCS 74(3): 303-307 (1997)), Collins et al. (J. Chromatography 445: 363-370 (1988)), Collins (J. Agric. Food Chem 37: 60-66 (1989)), and U.S. Pat. No 5,169,660, which are herein incorporated by reference.

(v) Avenanthramide Analysis

Total and individual avenanthramides can be determined by analytical HPLC by known methods. Individual avenanthramides can be identified by comparison of relative retention times and UV spectra with authentic standards. Quantification can be achieved by comparing peak areas with an external Avenanthramide A standard at 330 nm and expressed as Avenanthramide A weight equivalents.

(vi) Oat Extract Supplementation

Oat extract stock solution (100 mg/mL) was made in dimethyl sulphoxide (DMSO). The DMSO-oat extract solution was then diluted by MCDB-131 medium to make final concentrations of 4, 20 and 40 μg/mL to supplement HAEC. The concentration of DMSO in cultures media was 0.04%, which is tested to be not toxic to the cells (data not shown). Confluent monolayer HAEC in 24-well plates and 96-well plates were incubated with different concentrations of oat extract at 37° C. for 24 h.

(vii) Oat Extract Cytotoxicity

The oat extracts were dissolved in DMSO and diluted into MCDB-131 medium in different concentrations and incubated with HAEC for 24 h. The cytotoxicity of oat extracts at 4 μg/mL and 40 μg/mL in 0.04% DMSO (final concentration in medium) was tested using Trypan blue exclusion test.

(viii) Synthesis of Avenanthramide C (Av-C)

Avenanthramide-C (Av-C) can be synthesized, for example, by the techniques described in Peterson, D. M. J. Cereal Sci. 33: 115-129 (2001).

(ix) Fluorescent Labeling of Monocytes

U937 cells (American Type Culture Collection, Rockville, Md.), a human monocytic cell line, were used for monocyte-endothelial cell adhesion assay. This cell line has been used as a model for the blood-born monocyte in endothelial cell adhesion experiments. This cell line exhibits many characteristics of monocytes and is easy to use. A virtually unlimited number of cells can be prepared and are relatively uniform. This cell line has been an important tool in the investigation of the mechanisms involved in monocyte-endothelium attachment (DiCorleto et al. J Clin Invest. 75: 1153-1161 (1985)). The U937 cells were fluorescently labeled by incubating the cells (1×10⁷ cells/5 mL) with 5 μmol BCECF-AM/L [2′,7′-bis-(2-carboxyethyl)-5-(and- 6)-carboxy-fluorescein acetoxymethyl ester BCECF-AM (Molecular Probes, Eugene, Oreg.)] in PRM1-1640 medium for 30 min at 37° C. and 5% CO₂. BCECF-AM is a non-fluorescent lipophilic compound in which the ester bound is cleaved by intracellular esterase and becomes a highly charged fluorescent BCECF that is retained by viable cells. The BCECF-AM was prepared as a 1 g/L stock in DMSO and was stored at −80° C. After labeling, the cells were washed 3 times with 1% FBS in PBS to remove excess dye. Finally, cells were resuspended in MCDB-131 medium at a density of 5×10⁸ cells/L for the quantitative adhesion assay (Vaporciyan et al. J Immunol Methods. 159: 93-100 (1993)).

(x) U937 Cell Adhesion Assay

HAEC were cultured to confluence in 24-well plates and were treated with varying concentrations of oat extract for 24 hr. HAEC were then washed with PBS and activated with IL-1β (Endogen, Woburn, Mass.) 5 ng/mL for 6 h. BCECF-labeled U937 cells (1×10¹ were incubated with HAEC for 30 min at 37° C. After incubation, nonadherent cells were removed by washing each well 3 times with 1% FBS-PBS. The attached cells were lysed with 0.5 mL of 50 mmol/L tris buffer (pH 7.6) containing 0.1% sodium dodecyl sulfate. The fluorescence intensity of each well was measured with a Cytofluor (PerSeptive Biosystems, Framingham, Mass.) fluorescence multiwell plate reader set at excitation and emission wavelengths of 485 nm and 530 nm, respectively. With each set of experiments, a separate plate containing known numbers of U937 cells labeled with BCECF-AM was prepared for determination of a standard curve of fluorescence units per cell.

(xi) Adhesion Molecule Expression

Confluent HAEC in 96-well plates (Becton Dickinson Labware, Franklin Lakes, N.J.) were incubated with or without oat extract (4, 20 and 40 μg/mL) at 37° C. for 24 h. After the cells were washed with PBS, 5 ng/mL IL-1β (Endogen) was added to stimulate the cells at 37° C. for 6 h. Following decanting the medium, the cells were fixed with 1% paraformaldehyde at room temperature for 30 min. Enzyme linked immunosorbent assay (ELISA) was used to measure adhesion molecule expression. The plates were washed with PBS, blocked with 10% FBS for 1 hr. Monoclonal antibodies against human ICAM-1, VCAM-1 and E-selectin (PharMingen, San Diego, Calif.) were added at 2, 5 and 5 μg/mL respectively in 10% FBS/PBS forl h at room temperature. The secondary antibody, horseradish-peroxidase-conjugated anti-mouse IgG (Santa Cruz, Calif.) was added at 1:1000 dilution and incubated at room temperature for 1 hr. This was followed by addition of horseradish peroxidase substrate (Bio-Rad, Hercules, Calif.) and incubation for 1 hr. The plates were read at OD 405 nm by a plate reader (Bio-Tek Instruments, Winooski, Vt.).

(xii) Cytokine Expressions of HAEC

Confnluent HAEC in 24-well plates (Becton Dickinson Labware) were incubated without or with oat extract (4, 20 and 40 μg/mL) at 37° C. for 24 h. After the cells were washed with PBS, 5 ng/mL IL-1β (Endogen) was added to stimulate the cells at 37° C. for 24 hr. After centrifugation, the supernatants were collected and stored at −80° C. Sandwich ELISA was used to measure IL-6, IL-8 and MCP-1 expressions. Pro-Bind 96-well plates (Becton Dickinson) were coated with capture antibodies for IL-6, IL-8 (R&D System, Minneapolis, Minn.) and MCP-1 (PharMingen) overnight. After blocking with 10% FBS (Gibco) in PBS for 2 hr, the standards and samples were added and incubated at room temperature for 2 hr. Then biotinylated antibodies for IL-6, IL-8 (R&D System), MCP-1 (PharMingen) were added for 2 hr at room temperature. Avdin-peroxidase (Sigma) was used to amplify the reaction. Finally, horseradish peroxidase substrate (Bio-Rad) was added and incubated for 1 hr. The plates were read at OD 405 nm in a plate reader (Bio-Tek Instruments).

(xiii) Immunoblotting for p53, p21cip1, p27kip1, cyclin D1, pRb protein expression levels and pRb phosphorylation

Protein from 5×10⁴ cells was subjected to electrophoresis on 7.5-10% SDS-polyacrylamide gels and electro-transferred to nirocellulose membrane. Nonspecific binding was blocked by incubating with Tris-buffered saline (TBS) [137 mM NaCl, and 15 mM Tris-HCl (pH 7.6)] containing 5% non-fat milk (w/v) for 1 h at room temperature and then probed with primary antibody against p53, p21, p27, cyclin D1 and pRb overnight. After washing in TBS-Tween 20 buffer (0.075% v/v), the membranes were incubated with horseradish peroxidase-conjugated anti-IgG antibody. Proteins bound to the primary antibodies were detected with ECL detection reagents.

(xiv) Statistical Analysis

Data were analyzed using SYSTAT statistical package version 8.0 (Systat, 2001). Multiple comparisons of means were conducted by Tukey Post Hoc test. The overall oat extract effect was determined by ANOVA. Values are presented as means ±SD and p<0.05 is considered significant.

### Example 2

### Cytotoxicity Test

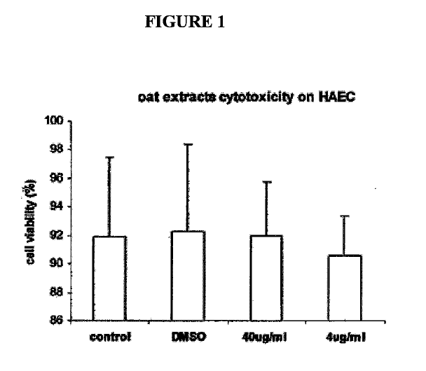

FIG. 1 shows oat extracts and DMSO cytotoxicity on HAEC. Confluent human aortic endothelial cells (HAEC) were incubated with 0, 4 and 40 μg/mL oat extracts and 0.04% DMSO for 24 h at 37° C. Cytotoxicity was measured by Trypan blue exclusion test. Data are the mean ±SD of 3 experiments, each performed in triplicate. *p<0.05, **p<0.01 compared with control. Oat extract had no cytotoxicity on HAEC up to the 40 μg/mL concentration tested. 0.04% DMSO in MCDB-131 medium solution showed also no toxicity on HAEC during 24 hr incubation.

### Example 3

### Effect of Oat Extract on Monocyte-HAEC Adhesion

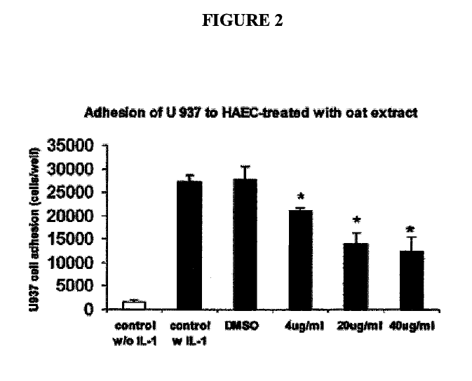

The effect of oat extracts on monocyte-endothelial cell adhesion is shown in FIG. 2. Confluent human aortic endothelial cells (HAEC) were incubated with 0, 4, 20 and 40 μg/mL oat extracts for 24 h at 37° C. The HAEC were then stimulated by interleukin (IL)-Iβ (5 μg/mL) at 37° C. for 6 h. A total of 10⁷ U937 cells were added onto HAEC and incubated at 37° C. for 30 min. The adhesion of U937 cells to HAEC was determined as described in Example 1. Data are the mean ±SD of 3 experiments, each performed in triplicate. *p<0.05, **p<0.01 compared with control. There was trivial adhesion of U937 to HAEC without IL-1, stimulation. Pre-treatments of HAEC with oat extracts or DMSO contributed little to that basal adhesion (data not shown). However, when HAEC was stimulated with 5 ng/mL IL-1β for 6 h, their adherence to U937 cells increased (p<0.01) (FIG. 2). Pretreatment of HAEC with oat extracts for 24 h before activation with IL-1β significantly reduced their adherence to U937 cells (p<0.05). Pretreatment of IL-1β-stimulated HAEC with 4, 20 and 40 μg/mL oat extracts reduced their adherence to U937 cells by 20%, 40% and 45%, respectively (FIG. 2).

### Example 4

### Effect of Oat Extract on the Expression of Adhesion Molecules

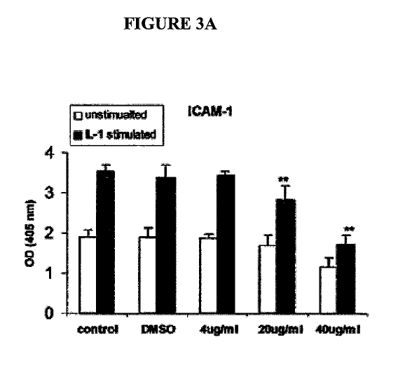

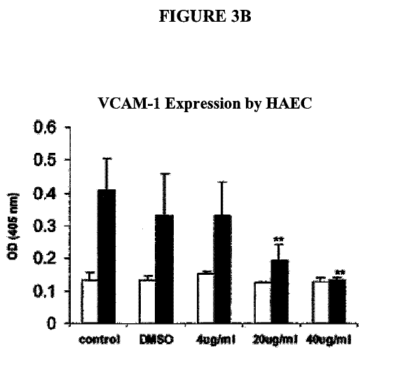

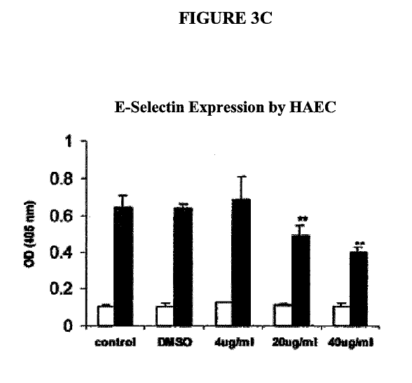

The effect of oat extracts on HAEC expression of adhesion molecules is shown in FIG. 3. Confluent human aortic endothelial cells (HAEC) were incubated with 0, 4, 20 and 40 μg/mL oat extracts for 24 h at 37° C. The HAEC were then stimulated by interleukin (IL)-Iβ (5 ug/mL) at 37° C. for 6 h. The expression of ICAM-1, VCAM-1 and E-selectin (A, B and C, respectively) on the cell surface was measured using ELISA as described in Example 1. Data are the mean ±SD of 3 experiments, each performed in triplicate. *p<0.05, **p<0.01 compared with control. The unstimulated HAEC constitutively expressed ICAM-1 was greater than VCAM -1 and E-selectin expressions (FIG. 3). With 5 ng/ml IL-1 stimulation for 6 h, the expression of adhesion molecules on the surface of HAEC increased significantly (P<0.01) (FIG. 3). Oat extract didn't significantly affect constitutive expression of adhesion molecules but significantly (P<0.05) inhibited IL-1β-stimulated expressions of ICAM-1 (FIG. 3A), VCAM-1 (FIG. 3B) and E-selectin (FIG. 3C). Pretreatment HAEC with 20 μg/mL and 40 μg/mL oat extracts significantly reduced the expressions of adhesion molecules on IL-1β-stimulated HAEC compared with the untreated cells (P<0.05). Pretreatment with 40 μg/mL oat extract resulted in the most effective inhibition of adhesion molecules expression. The expressions of ICAM-1 and VCAM-1 in the IL-1β-stimulated HAEC were nearly abrogated by preincubation of HAEC with 40 ug/mL oat extracts (FIG. 3).

Following IL-1 β stimulation of HAEC, the expressions of ICAM-1 and VCAM-1 increased several folds and pretreatment of HAEC with oat extracts dose-dependently inhibited the expressions of these adhesion molecules leading to a decreased adhesion of U937 cells to IL-1β-stimulated HAEC. The highest dose of oat extracts 40 μg/mL totally abrogated the stimulatory effect of IL-1β.

The study shows, oat extracts inhibited IL-1β stimulated HAEC adhesion to U937, but not basal unstimulated adhesion. It was also found that IL-1β-activated HAEC expressions of three important adhesion molecules was inhibited while the constitutive expression was unaffected by oat extracts treatment. Therefore, the reduction of HAEC adhesion to U937 by oat extracts appears to be mediated by the suppression of adhesion molecules expressions by HAEC. The secretions of IL-8, MCP-1 and IL-6 by HAEC were also affected by oat extracts treatment suggesting that oat extract may interfere with some common activation mechanism responsible for the induction of these active molecules. Although the effect of oat extract on NF-κB inhibition was not examined, the results suggest oat extract treatment may have influenced this signaling pathway in HAEC. It is well established that the nuclear transcription factor NF-κB binding sites in the promoters of ICAM-1, VCAM-1 and E-selectin genes are necessary for their induction via cytokines (Whelan et al. Nucleic Acids Res. 19: 2645-53 (1991); Collins et al. Faseb J. 9: 899-909 (1995)). Activation of NF-κB also results in expression of mRNA of a variety of pro-inflammatory mediators such as IL-8, IL-6, MCP-1 and adhesion molecules (Christman et al. Intensive Care Med. 24: 1131-8 (1998)). Because some evidence support the role of ROS in NF-κB activation (Schreck et al. Free Radic Res Commun. 17: 221-237 (1992)), antioxidants have been investigated as inhibitor of NF-κB activation. Direct treatment with oxidants such as H₂O₂ activated NF-κB. While antioxidants have been shown to inhibit NF-KB activation in vitro (Van den Berg et al. Br J Nutr. 86 Suppl 1: S121-7 (2001)). Up-regulation of endogenous oxidant defenses has been demonstrated to suppress NF-κB activation (Mirochnitchenko et al. J Immunol, 156: 1578-1586 (1996)). Activation of NF-κB in vitro has been shown to be inhibited by a variety of antioxidants including vitamin E derivatives, pyrrolidine dithiocarbamante (PDTC), N-acetylcysteine (NAC), ascorbic acid and etc. (Christman et al. Intensive Care Med. 24: 1131-1138 (1998)). Therefore, the mechanism for antioxidant inhibition of pro-inflammatory cytokines, chemokines, adhesion molecules and HAEC adhesions to monocyte potentially were mediated by inactivation of NF-κB signaling pathway.

### Example 5

### Effect of Oat Extracts on the Production of Cytokines

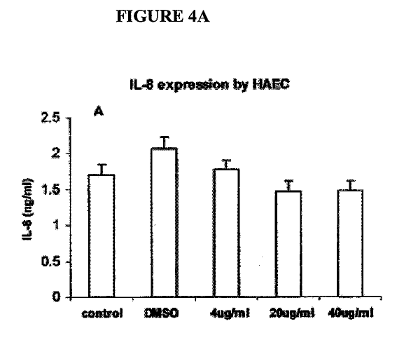

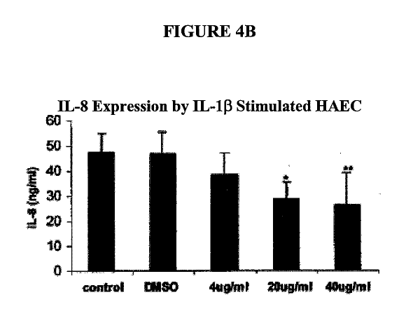

The effects of oat extracts on HAEC expression of IL-8, IL-6, and MCP-1 are shown in FIGS. 4, 5, and 6, respectively. FIG. 4 shows the effect of oat extracts on HAEC expression of IL-8. Confluent human aortic endothelial cells (HAEC) were incubated with 0, 4, 20 and 40 μg/mL oat extracts for 24 h at 37° C. The HAEC were then stimulated by interleukin (IL)-Iβ (5 μg/mL) at 37° C. for 24 h. The expression of IL-8 on the cell surface was measured using ELISA as described in Example 1. Blank bars shown in FIG. 4A represent constitutive expressions. Shaded bars shown in FIG. 4B represent IL-1β stimulated expressions. Data are the mean ±SD of 3 experiments, each performed in triplicate. *p<0.05, **p<0.01 compared with control.

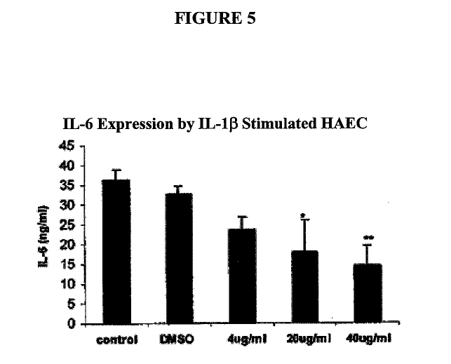

FIG. 5 shows the effect of oat extracts on HAEC expression of IL-6. Confluent human aortic endothelial cells (HAEC) were incubated with 0, 4, 20 and 40 μg/mL oat extracts for 24 h at 37° C. The HAEC were then stimulated by interleukin (IL)-Iβ (5 μg/mL) at 37° C. for 24 h. The expression of IL-6 on the cell surface was measured using ELISA as described in Example 1. Shaded bars shown in FIG. 5 represent IL-1β stimulated expressions. The IL-6 baseline was too trivial to be detected. Data are the mean ±SD of 3 experiments, each performed in triplicate. *p<0.05, **p<0.01 compared with control.

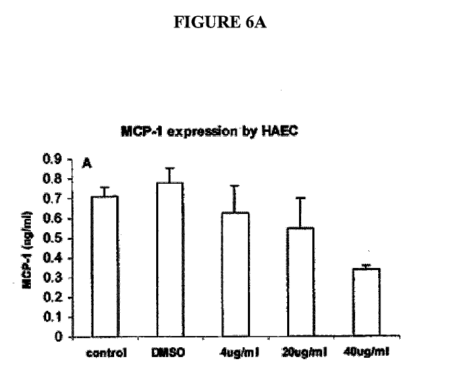

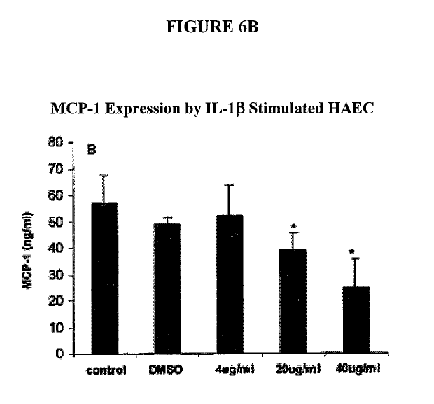

FIG. 6 shows the effect of oat extracts on HAEC expression of MCP-1. Confluent human aortic endothelial cells (HAEC) were incubated with 0, 4, 20 and 40 μg/mL oat extracts for 24 h at 37° C. The HAEC were then stimulated by interleukin (IL)-Iβ (5 μg/mL) at 37° C. for 24h. The expression of MCP-1 on the cell surface was measured using ELISA as described in Methods. Blank bars shown in FIG. 6A represent constitutive expressions. Shaded bars shown in FIG. 6B represent IL-1β stimulated expressions. Data are the mean ±SD of 3 experiments, each performed in triplicate. *p<0.05, **p<0.01 compared with control.

The production of IL-8, IL-6 and MCP-1 by HAEC significantly increased with IL-1β stimulation compared with unstimulated cells (P<0.01). There was no detectable levels of IL-6 baseline, and very low level of IL-8 and MCP-1 were present before activation of HAEC with IL-1β(FIGS. 4A & 6A). Supplementation of HAEC with oat extracts showed no effect on the IL-8 and IL-6 expressions in unstimulated cells. However, pretreatment of unstimulated HAEC with 40 μg/mL oat extract significantly inhibited MCP-1 production (P<0.05) (FIG. 6A). Pretreatment HAEC with oat extract 20 μg/mL and 40 μg/mL decreased the production of IL-8 (FIG. 4B), IL-6 (FIG. 5) and MCP-1 (FIG. 6B) (P<0.05) following stimulation with IL-1β.

In a confluent HAEC monolayer culture, the constitutive expression of IL-8 is very low, however, its expression is increased by 30 fold following the activation of HAEC. Supplementing HAEC with oat extracts dose-dependently inhibited IL-8 production in the IL-1β activated HAEC.

The production of MCP-1 by unstimulated HAEC in the present study was low. However it was remarkably increased with IL-1β stimulation (FIGS. 6A & B). Supplementing HAEC with oat extracts dose-dependently inhibited the MCP-1 production by the activated cells. However, only highest dose of oat extract (40 μg/mL) was effective to reduce its production to a significant level in the inactivated cells. Monocyte recruitment to the site of inflammation is believed to be a major determinant of atherosclerotic lesion size and complexity (Reape et al. Atherosclerosis. 147: 213-25 (1999)). Thus, the findings that oat extracts dose-dependently decreased MCP-1 production by IL-1β stimulated HAEC may have important implications in terms of the potential role of oat in reducing recruitment and transmigration of monocytes across the endothelium.

In the HAEC monolayer, the basal production of IL-6 was too low to be detected. However, with IL-1β stimulation for 24 hr, the production of IL-6 by HAEC was increased significantly, and pre-incubation of HAEC with oat extracts dose-dependently reduced the IL-6 production in IL-1β stimulated HAEC. Therefore, suppression of IL-6 production in activated HAEC by oat extracts provides another potential protective mechanism by which consumption of oat may contribute to the reduction of risk of atherosclerosis, through inhibiting SMC proliferation, migration and atherosclerotic plaque formation.

Recruitment of monocytes to the intima is one of the earliest events in the formation of an early lesion of atherosclerosis (Libby, P. J Intern Med. 247: 349-358 (2000)). In response to inflammatory stimuli such as IL-1β, TNF-α the activated endothelium recruits leukocytes to the site of activation by production of chemokines such as IL-8, MCP-1 and IL-6, then selectively expresses adhesion molecules to capture the immune cells (Vanhee et al. Cell Immunol. 155: 446-456 (1994)). The observation that oat extracts dose-dependently reduced production of these cytokines in the IL-1β stimulated HAEC is novel and provides yet another potential mechanism by which oat may reduce the risk of atherosclerosis in vivo.

### Example 6

### Effect of Phenolic Compounds on FBS-Induced Cell Proliferation of HSMC

[³H] thymidine incorporation into DNA was used to determine the effect of Avenanthramide on cell proliferation. Synthetic Av-C was used in this experiment. Human aortic smooth muscle cells (HSMC) were seeded into 24-well plate at equal density. After reaching to 80% confluency, the cells were synchronized to quiescent condition by serum starvation for 48 h. Cells were then stimulated with 10% of FBS in the absence or presence of different concentration of Av for 24 h. During the last 4 h of incubation, 1.0 μCi/mL of [³H] thymidine was added to each well. DNA was precipitated with 10% trichloroacetic acid and solubilized with 0.1N sodium hydroxide and counted in a scinitillation counter. Treatment of HSMC with Avenanthramide inhibited serum-induced DNA synthesis and thus cell proliferation (FIG. 7). The inhibitory effect of Avenanthramide was concentration-dependent. At concentration of 120 μM, Avenanthramide inhibited more than 50% of cell proliferation without significant cytotoxity as determined by trypan blue cell viability assay.

The cell numbers were counted to verify that the results observed with the [³H] thymidine incorporation were reflective of changes in cell growth. HSMC or A10 cells (rat embryonic aortic smooth muscle cell) were seeded into 6-well plate at a density of 0.5×10⁴/well. Four hr later 120 μM of avenanthramide were added to each well. At different time points (2d, 3d and 4d) the cells were trypsinzied and the total cell number were counted using a hemocytometer. Trypan blue exclusion test was carried out to determine the cell viability.

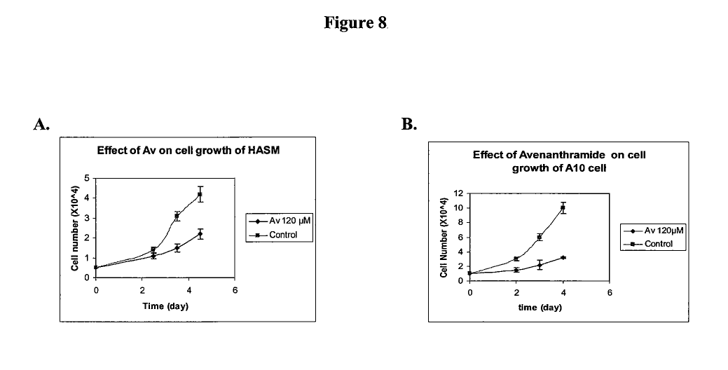

As shown in FIG. 8, 10% FCS caused a rapid increase of cell number, the doubling time for HSMC and A10 are calculated to be 28 hr and 38 hr, respectively. The addition of 120 μM Avenanthramide attenuated this cell number increase and their doubling time increased to more than 48 hr. This result is consistent with the result obtained from [³H] thymidine incorporation. Avenanthramide seemed to have more inhibitory effect for A10 cell than for HSMC. When Avenanthramide was removed from cell culture medium, the cells proliferation restarted again (data not shown), indicating that the inhibitory effect was reversible.

### Example 7

### Effect of Phenolic Compounds on NO Production

In addition to its vasodilating feature, NO has antiatherosclerotic properties, such as prevention of platelet aggregation, leukocyte adhesion, smooth muscle cell proliferation, and the expression of genes involved in atherogenesis. In this study, the human aortic smooth muscle cells (HSMC) and human aortic endothelia cells (HEC) were used to determine if avenanthramides can influence the NO production level. Synthetic Av-C was used in this experiment. The quantification of NO production was performed by the 4,5 diaminofluorescein (DAF-2) fluorescence assay. Briefly, cells were seeded into 24-well plate and let them grow to 90% confluent condition. The cells were pretreated with different concentrations of Avenanthramide for 24 h. The culture medium was then changed to PBS containing 100 μM L-arginine, 1 μM bradykinin and L-NAME. Then 0.1 μM of DAF-2 was added to each well. After another 2 h of incubation, 200 μL of supernatants were transferred to black microplates and the fluorescence was measured with a fluorescence microplate reader calibrated for excitation at 485 nm and emission at 538 nm.

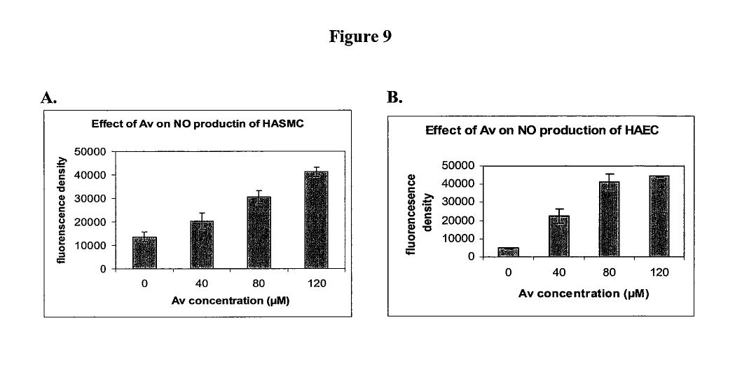

As shown in FIG. 9, Avenanthramide dose-dependently and significantly induced NO production of both HSMC and HAEC. For these two cells, at concentration of 120 μM, Avenanthramide respectively induced 3.0 folds and 8.8 folds increase in NO production compared with control (P<0.05).

### Example 8

### Effect of Phenolic Compounds on eNOS mRNA level

Since NO is catalyzed by endothelium nitric oxide synthases (eNOS), the role of Avenanthramide in induction of eNOS expression level by real time PCR was examined. Total mRNA was extracted using RNesy Mini Kit (QIAGEN) and quantified spectrophotometrically at 260 nm. Twenty μl of first-strand cDNA was synthesized from 1.5 μg of total RNA by using 300 ng of random hexanucleotide primers and 200u of SuperScript II (Invitrogen) at 42 C. for 1 h followed by heat inactivation of reverse transcriptase at 70 C. for 15 min. Fifty μL of PCR reaction mixture contained 5 μL cDNA, up- and down-stream primers (200 nM) and 25 μL of SYBR Green was set up into the ABI 4400 to perform the amplification for 40 cycles with denaturation at 95 C. for 30 sec, annealing at 60 C. for 1 min and extension at 72 C. for 1 min. Primers designed based on the published gene sequences were synthesized from Tufts University Core Facility. The primer sequences used for eNOS were:

| | |
|---|---|
| 5′-ATCCTGGCAAGCCCTAAGACC-3′ | (upstream) |
| and | |
| 5′-TGGTAGCGTTTGCTGATCCCG-3′ | (downstream). |

Beta-actin was used as an internal control with the primers:

| | |
|---|---|
| 5′-TTGTAACCAACTGGGACGATATGG-3′ | (upstream), |
| 5′-CACAATGCCAGTGGTACGACC-3′ | (downstream). |

Beta-actin mRNA expression level as an internal control. As shown in FIG. 4, compare with control, treatment with Avenanthramide increased the eNOS mRNA expression level. The enhancing effect is dose-dependent and the pattern is consistent with the results from NO production (FIG. 3).

TABLE 1
Effect of Phenolic Compounds on eNOS mRNA expression level

| Av. | eNos Average | | ΔCt | Fold |
|---|---|---|---|---|
| Con. (μM) | CT | β-actin CT | eNOS-β-actin | Change |
| 0 | 35.6 ± 0.21 | 27.9 ± 0.25 | 7.67 ± 0.33 | 1 |
| | | | | (0.67-1.33) |
| 40 | | 28.8 ± 0.34 | 7.0 ± 0.51 | 1.56 |
| | | | | (1.22-1.9) |
| 80 | | 29 ± 0.33 | 6.41 ± 0.48 | 2.39 |
| | | | | (2.1-2.7) |

### Example 9

### Effects of Phenolic Compounds on Phosphorylation of pRB in A10 Cells

The effect of phenolic compound treatment on cell cycle regulatory molecules involved in the G1 phase of the cell progression was analyzed. pRB, the product of the retinoblastoma tumor suppressor gene, is a major negative regulator of cell proliferation and operates in the middle to late G1 phase of the cell cycle. Phosphorylation of pRB inactivates the growth inhibitory function of pRB. To elucidate the mechanism by which the phenolic compounds inhibit the cell cycle progression, the changes in pRB phosphorylation were measured after the FBS stimulation in the presence or absence of different concentrations of substantially purified Avn-C.

At 80% confluence, A10 cells seeded into 100 mm culture dishes and were treated with different concentrations of Av (0, 40, 80 and 120 μM) for 24 h. Following Avn treatment, the total cell extracts were harvested with 2× protein sample buffer [0.14 M Tris/HCl (pH 6.8), 22.4% glycerol, 6% SDS, 0.02% bromophenoxy bromide, and 10% β-mercaptoethanol] by scraping the cells. The genomic DNA is sheared by passing through a 21-gauge syringe several times. The homogenized cell lysates were boiled for 5 min and centriftiged at 12,000 rpm for 5 min. Protein half life studies are performed as described before. Briefly, cells were treated with Avn for 4-6 h. Cycloheximide (20 μg/mL) was then added to inhibit further protein synthesis. Cells were harvested in 2× protein sample buffer at 0, 30, 60 and 120 min after cycloheximide addition. Protein samples were analyzed by Western blot.

At 50% confluence (exponentially growing phase), A10 cells were seeded into 100 mm culture dishes and were treated with different concentrations of Avn for 48 h. Cells were then harvested by trypsin/EDTA treatment, washed with PBS and fixed with 70% ethanol at 4° C. for 1 hr. Prior to analysis, cells were washed again with PBS and stained with 1.0 mL of propidium iodide (PI) solution containing 25 μg/mL PI and 10 μg/mL RNase in PBS at 4° C. for 30 min in the dark. Cell cycle distributions were then analyzed by flow cytometry using the fluorescence-activated cell sorting (FACS) analysis. To calculate percentage of cells in respective phases of the cell cycle the DNA content frequency histograms were deconvoluted. Based on the DNA content, the cell cycle can be distinguished from G1 to S and G2-M phase.

As shown in Example 6, Avn inhibited FBS-induced cell proliferation as determined by [3H] thymidine incorporation and cell number counting. This Example analyzed the effect of Avn on cell cycle distribution by flow cytometry. In control A10 SMC (not treated) in growing phase, about 40% of the cells were in S-phase while 49.7% of cells were in G0/G1-phase. When the growing cells were treated with 120 μM of Avn for 48 h, the population of cells in S-phase decreased to 25.7% while the population of cells in G0/G1-phase increased to 66.9%. This result indicated that Avn inhibited the cell proliferation of SMC and arrested the cell cycle at G1-phase.

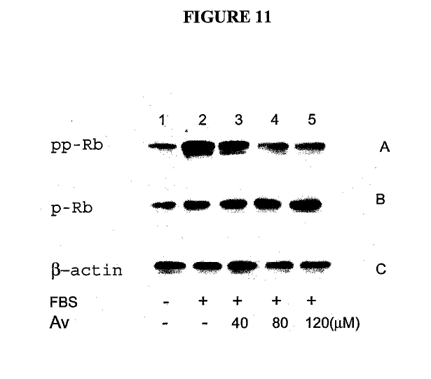

Based on the above findings that treatment of A10 cells with Avn arrests the cell cycle at G1 phase, the effect of Avn treatment on cell cycle regulatory molecules involved in G1 phase of the cell cycle progression were analyzed. pRB, the product of the retinoblastoma tumor suppressor gene, is a major cell cycle regulator and its hyperphosphorylation (ppRb) is necessary for G1-S phase transition. To elucidate the mechanism by which Avn inhibits the cell cycle progression, the changes in pRB phosphorylation status were measured using anti-phospho-pRb specific antibody. FIG. 11 is a Western blot showing the effects of Avn on pRB phosphorylation. Av inhibits pRb phosphorylation in A10 cells stimulated with FBS. Cells were seeded into 60 mm Petri dishes and cultured in 10% FBS-DMEM until 70% confluent. The media were changed into FBS-free DMEM and cultured for another 24 h to get the cells synchronized. The cells were then stimulated with 10% FBS in the absence or presence of different concentrations of Av. After 20 h, total cell lysates were prepared and subjected to SDS-PAGE followed by Western immunoblotting. Membranes were probed with anti-ppRb, pRb and beta-actin antibodies followed by peroxidase-conjugated secondary antibodies, and visualized by ECL detection system. After stimulation with serum, the amount of hyperphosphorylated forms markedly increased after 20 h adding FBS. Substantially purified Avn-C dose-dependently inhibited pRB phosphorylation. This inhibition was only due to the inhibition of phosphorylation, but not due to the inhibition of the total pRb protein level. In quiescent cells as shown in panel A of FIG. 11, most of pRb is hypophosphorylated (lane 1). After the stimulation with serum, the amount of hyperphosphorylated forms (ppRb) markedly increased after 20 h (lane 2). Avn treatment significantly and dose-dependently attenuated the serum-induced pRb phosphorylation (lane 3-5). To ensure that the observed effect is not due to the Avn decreasing the total amount of pRb, the expression of the total amount of was assessed by anti-pRb antibody, which recognizes both hyper- and hypophosphorylated pRb. As shown in panel B of FIG. 11, the expression of the total amount of pRb did not change upon serum stimulation and Avn treatment, which indicate that the inhibitory effect of Avn was only due to the inhibition of phosphorylation of pRb and not due to the inhibition of the total pRb protein level. Panel C of FIG. 11 shows the β-actin expression as loading control.

### Example 10

### Effects of Phenolic Compounds on Cyclin D1 Expression in A10 Cells

The pRB phosphorylation is brought about by a group of serine/threonine kinases, cyclin-dependent kinase (CDK). Cyclin D is expressed upon mitogen stimulation and associates with CDK to form active cyclin D/CDK complex, which is responsible for the phosphorylation of pRB in G1 phase. As shown in Example 9, pRB phosphorylation is inhibited by Avn treatment. Since cyclin D upregulates CDK activity, the effect of Avn on serum-stimulated expression of cyclin D1 was explored.

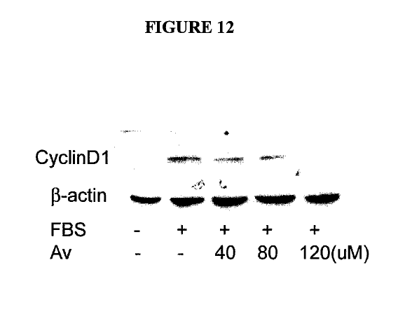

Cells were treated with 10% FBS in the presence and absence of substantially purified Avn-C as described above. 20 h after the addition of serum to quiescent A10 cells, the amount of cyclin D1 was measured by Western blotting using polyclonal anti-cyclin D1 antibody. β-Actin expression level was used as a loading control. As shown in FIG. 12, in quiescent A10 cells, the amount of cyclin D was too little to be detectable. Upon 20 h of stimulation with 10% FBS, the amount of cyclin D increased significantly, indicating cells change from a quiescent state to a proliferating state. Thus, Avn treatment was shown to attenuate the FBS-induced cyclin D expression, and this inhibitory effect is dose-dependent.

### Example 11

### Effect of Phenolic Compounds on p53 Expression Level

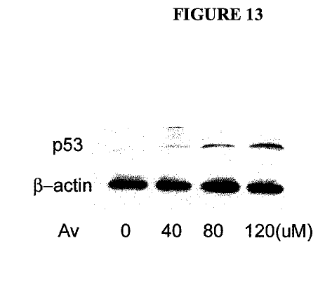

Induction of the tumor suppressor p53 has been implicated in the control of cell growth of SMC, and some antioxidants in green tea or red wine can increase the p53 protein expression level. Therefore, the effect of substantially purified Avn-C treatment on p53 protein levels was explored. A10 cells were seeded into 100 mm Petri dishes and grown in 10% FBS culture medium until 70% confluence. Cells were either left untreated or treated with different concentrations of Avn (0-120 uM), and the cells were harvested after 24 h. Whole cell lysates were prepared and subjected to Western blot analysis for p53 and β-actin levels. Avn produced a dose-dependent increase in p53 protein levels with 120 uM Avn producing highest fold induction of the concentrations tested (FIG. 13).

### Example 12

### Effect of Phenolic Compounds on p21 Expression Level

Cyclin-dependent kinase (cdk) inhibitors play important roles in cell cycle progression. P21cip1 is the main target of p53 medicated cell cycle inhibition. Upregulation of p21cip1 has also been shown to be one of the main mechanisms involved in the prevention of pRb phosphorylation, which causes cell cycle inhibition. The effect of Avn on this CDK inhibitor p21cip1 protein expression was determined by Western blotting. A10 cells were synchronized by growning in FBS-free medium for 24 h. The cells were then stimulated with 10% FBS in the absence or presence of different concentrations of substantially purified Avn-C. After 20 h, total cell lysates were prepared and subjected to SDS-PAGE followed by Western blot analysis to detect p21cip and p27kip. β-Actin expression was used as loading control. As shown in FIG. 14, quiescent cells expressed relatively high levels of p21. Upon FBS stimulation, p21 was down-regulated and cells entered into a proliferating cycle. When quiescent cells were stimulated with FBS in the presence of Avn, the p21 expression level increased significantly compared to control and this inducible effect of Avn was dose-dependent with the highest level at 120 μM. The observed induction in p21cip1 protein levels by Avn is not due to a change in protein loading as confirmed by probing the same membrane with beta-actin antibody (FIG. 14).

p27Kip1 is another member of the CIP/KIP family of cdk inhibitors that negatively regulates cyclin-cdk complexes. However, as shown in FIG. 14, Avn has no significant effect on p27Kip1 protein expression in A10 cells.

### Example 13

### Detecting the Half Life of p53 Protein

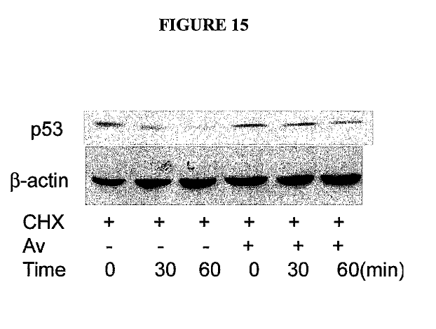

P53 protein level is mainly regulated at a posttranslational level, usually via control of protein stability. To determine whether increased protein stability might play a role in the increased p53 levels induced by Avn treatment observed in this study, the change in half life of p53 protein in Avn-treated and nontreated cells was explored. A10 cells were treated with 0 or 80 uM of substantially purified Avn-C for 6 h, then 15 μg/mL cycloheximide was added to block further protein synthesis. Whole cell extract was prepared at different time points (0, 30 and 60 min.) and p53 protein levels were measured by Western blot. In non-Avn treated control cells, the amount of p53 decreased to a little less than 50% at 30 min, and at 60 min, it decayed to less than 10% of the low basal level. This data indicated that the half life of p53 is around 20-30 min, which is consistent with values seen in other cells for wild-type p53 protein. As shown in FIG. 15, treatment with substantially purified Avn-C induced p53 levels as compared with nontreated control values (Avn+, 0min via Avn−, 0min) is consistent with our findings discussed above. Avn treatment led to significant stabilization of p53 protein. More than half of the p53 protein remained after 1 h of cycloheximide treatment. Thus, exposure of A10 cells to substantially purified Avn-C causes an increase of half-life of p53, which might account for the increase in p53 levels by Avn treatment.

### Example 14

### Methyl Ester of Avenanthramide (CH₃-Avn-C)

(i) Synthesis Methyl Ester of Avenanthramides

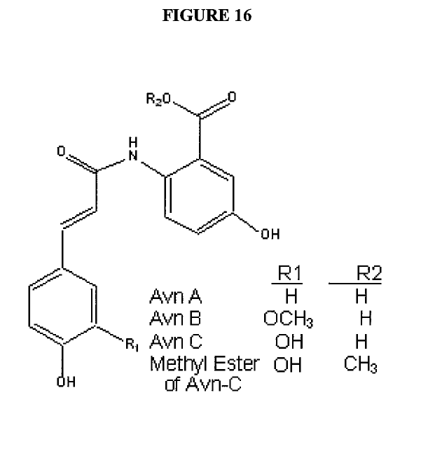

The methyl ester form of avenanthramide-C(CH₃-Avn-C) (FIG. 16) was synthesized and tested for its efficacy on the inhibition of vascular smooth muscle cell (VSMC) proliferation as well as for its cytotoxicity. The methyl ester form of avenanthramide-C can be synthesized using the Fieser Method (See, for example, Fieser, L. F. and Fieser, M., Reagents for Organic Synthesis. 1967, New York, N.Y.: Wiley.). Briefly, acidic methanol was made by adding about 5 ml of acetyl chloride to 100 ml of absolute methanol and then the Avns were introduced and stirred overnight at room temperature. The solvent was removed under vacuum and methylated Avn washed several times with methanol. As shown below, the substitution of OH-moiety on the A-ring of avenanthramide-C (Avn-C) increases the potency of avenanthramide-C by more than 10-fold.

A detailed exemplary method of synthesis of (CH₃-Avn-C) is described. Slowly add 2.5 ml acetyl chloride to 47.5 mL of anhydrous methanol. To this solution add the AvnC and allow approximately 24 hours at room temperature protected from light, monitoring the reaction by TLC (silica gel 60 F254) using an ethyl acetate/hexane/acetic acid (70:25:5) mobile phase. The reaction solution is then dried under vacuum (rotory vaporation) and then the product is washed several times with acetone. Resuspend the product in a minimal volume of 100% MeOH and then dilute to 20% MeOH with H₂O and load to a Fisher (tradename) PrepSep C-18 column preequilibrated with 20% MeOH. W ash the column with 5 volumes of 20% MeOH followed by 40% MeOH then elute the methyl ester with 100% MeOH to obtain the pure AvnC Methyl ester in approximately a 60% yield. Product was analyzed by TLC and LC/MS.

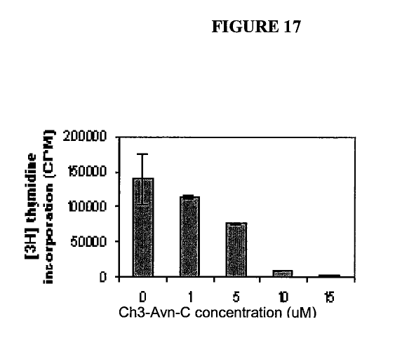

(ii) Effects of Methyl Ester of Avenanthramides on FBS-Induced Cell Growth of A10 Cells

[³H] thymidine incorporation. The effect of CH₃-Avn-C on thymidine incorporation was detected in A10 cells stimulated with 10% FBS. A10 cells were seeded in triplicate in 24-well plates at a concentration of 5×10⁴ cells/well. After 80% of confluence, cells were serum-starved for 24 h followed by serum stimulation in the presence of different concentrations of CH₃-Avn-C (0-15 μM) for 28 h. Cells were pulsed with 1.0 μ/Ci/mL of [³H] thymidine during the last 4 h before the end of the incubation. After washing the cells with cold PBS, DNA was precipitated with 10% trichloroacetic acid, solubilized with 0.1N sodium hydroxide, and counted in a scintillation counter. Each concentration point was done in triplicate wells, and at least three independent experiments were performed. Results are expressed as the mean ±SEM. As shown in FIG. 17, DNA synthesis was totally abolished by 15 μM CH₃-Avn-C.

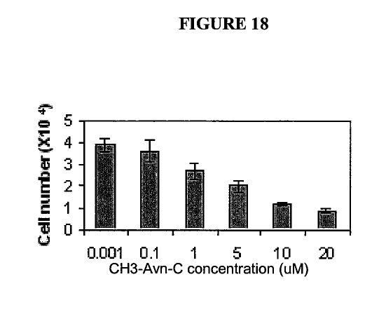

Cell growth assay: A10 cells were seeded in triplicate into 24-well plates. After 4 h the cells were attached, and then different concentrations of CH₃-Avn-C (0-20 μM) were added and cultured for 48 h. The cells were trypsinzied and the total cell number was counted using a hemocytometer. Trypan blue exclusion test was carried out to determine cell viability. As seen in FIG. 18, cell number was reduced by 80% with 20 μM of CH₃-Avn-C.

Effect of CH₃-Avn-C on Reactive Oxygen Species (ROS) Production of Monocytic Cells.

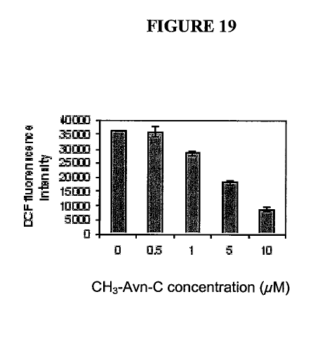

As shown in FIG. 19, the intracellular concentration of ROS in U937 cells, as measured by DCHF, was increased by hydrogen peroxide treatment. Pretreatment of U937 cells with Avn for 18 h significantly inhibited hydrogen peroxide-induced ROS production. Pretreatment of H₂O₂-stimulated U937 cells with 1, 5, and 10 μM of Avn reduced the ROS production by 22, 50, and 76%, respectively, indicating the inhibitory effect is dose-dependent.

Effect of CH₃-Avn-C on cell proliferation and cell viability. As shown in FIG. 18, as low as 10 μM of CH₃-Avn-C was effective at reducing proliferation of rat aortic smooth muscle cells (A10) by 73%, which is significantly higher than the effect of 120 μM of the non-methylated form of Avn-C, which only resulted in 50% reduction in cell proliferation. CH₃-Avn-C at a concentration of 10 μM has no cytotoxic effect on A10 cells (data not shown). This differential effect of CH₃-Avn-C is potentially related to its increase in lipophilicity due to the methyl ester group; thus, it is readily taken up by the cells. CH₃-Avn-C is better and more efficiently incorporated into the cell and it modulates cell signaling molecules. At this low concentration, CH₃-Avn-C also works through its antioxidant activity. It inhibits cell proliferation by modulating the cell cycle regulating molecules such as cyclin D₁, pRB, P21, and P27, as shown in the Examples above.

### Example 15

### Phenolic Compounds and Methyl Ester of Avenanthramide (CH₃-Avn-C) Inhibit Colon Cancer Cell Proliferation

In this Example, the effect of Avns on cell proliferation of several human colon cancer cell lines including Caca2, HCT116, and HT29 cells was explored. One important property of CaCo2 cells is that they can become differentiated by incubating confluent cells for 7-10 days (Rousset, M., The human colon carcinoma cell lines HT-29 and Caco-2: two in vitro models for the study of intestinal differentiation. Biochimie, 1986. 68(9): p. 1035-40), which is a good model to study the effect of chemotherapeutic agents on cancer vs. its normal phenotype cells. Alkaline phosphatase (ALP) activity, a marker of enterocytic differentiation, progressively increases between day 7-13 in culture, which correlates well with differentiation. The growth status of CaCo2 from growth to differentiation is also associated with significant changes in the expression and secretion of insulin-like growth factor-II (IGF-II) and IGF binding protein (IGFBP) (Singh, P., et al., Proliferation and differentiation of a human colon cancer cell line (CaCo2) is associated with significant changes in the expression and secretion of insulin-like growth factor (IGF) IGF-II and IGF binding protein-4: role of IGF-II. Endocrinology, 1996. 137(5): p. 1764-74).

As shown below, Avns significantly inhibit the proliferation of growing phase of Caco2 cells but have little effect on the differentiated CaCo2 cells, which exhibit normal characteristics of colonic epithelial cell. CH₃-Avn-C has a stronger inhibitory effect (IC₅₀ of 30 μM) compared with Avns oat. The data showed that HCT cells, which are COX2 negative, are more sensitive to Avns than the other colon cancer cell lines, which are COX2 positive. These data indicate that the growth inhibitory effects of Avns is independent on COX 2 pathway. Therefore, the mechanisms of Avns for inhibiting colon cancer cell proliferation is different from that of other polyphenols, such as curcium or EGCG, whose inhibitory effects are related to COX expression level.

(i) Materials and Methods for Colon Cancer Proliferation Experiments

Materials: All human colon cancer cell lines and McCoy's 5 medium were obtained from American Type Culture Collection (ATCC, Rockville, Md.). FBS and DMEM medium were purchased from GIBCO (Grand Island, N.Y.). NS398 was obtained from Sigma-Aldrich CO. (St. Louis, Mo.). Anti-COX-1 and -2 antibodies were purchased from Santa Cruz Biotechnology, Inc. (Santa Cruz, Calif.). Anti-phospho-p65 NFkB, anti-tublin, anti-rabbit, and anti-mouse antibodies were purchased from Cell Signaling Technology (Beverly, Mass.). TNFα and CellTiter 96 AQueous One Solution Cell Proliferation Assay were obtained from Promega Corp. (Madison, Wis.). Western blotting supplies were obtained from BioRad Laboratories (Hercules, Calif.). Prostaglandin E2 EIA kit was purchased from Cayman Chemical Company (Ann Arbor, Mich.). Avns oat extract and CH₃-Avn-C were prepared as described above.

Cell culture: HT-29 human colon adenocarcinoma cancer cells were cultured in McCoy's 5A medium, LS-174T and Coco2 human colon adenocarcinoma cancer cells were cultured in DMEM. Both medium contained 10% FBS and 1% penicillin/streptomycin. Cells were incubated at 37° C. in a humidified atmosphere of 5% CO₂. To induce differentiation, confluent Caco2 cells were further cultured for another 10-14 days. Otherwise, cells were maintained in exponentially growing phase by subculture twice a week at a density of 2×10⁵/10 cm culture dish. Avns oat extract and CH₃-Avn-C were dissolved in dimethyl sulfoxamine (DMSO) and added to the cell culture medium with a maximum final DMSO concentration of 0.2%, which showed no cytotoxicity and cell proliferative effect as determined by trypan blue exclusion assay.

Cell Proliferation Assay: Proliferation was measured using CellTiter 96 AQueous One Solution Cell Proliferation Assay. Cells were seeded into 96-well plates at a density of 3000 cells/well/100 μL and incubated for 6 hr for cell adhesion. Then cells were treated with different concentrations of Avns oat extract and CH₃-Avn-C. After 48 hr of treatment, the cell viability was measured according to the manufacture's instruction. Data were expressed as the percentage of untreated cells.

Western Blot Assay: HT-29 cells were seeded into 60 mm culture dishes and cultured to 70% confluence. Medium was changed from 10% to 1% FBS medium to minimize the effects of other growth-promoting substances normally contained in serum. For COX-2 expression studies, the cells were pre-treated with different concentrations of Avns oat extract and CH₃-Avn-C for 1 hr followed by stimulation with 40 ng/ml of TNF-α for 23 hr. The medium was collected and kept at −80° C. for further analyzing prostaglandin E2 (PGE2) production. For analyzing the NFkB phosphorylation, the cells were pre-treated with different concentrations of Avns oat extract and CH₃-Avn-C for 23 hr followed by stimulation with 40 ng/ml of TNF-α for 20 min. For determining the COX1 expression level, the cells were treated with different concentrations of Avns oat extract and CH₃-Avn-C for 24 hr. To get the whole cell lysate for western, cells were harvested in 2× protein sample buffer by scraping the cells. The genomic DNA was sheared by passing it through a 21-gauge syringe needle several times. The homogenized cell lysates were boiled for 5 min and centrifuiged at 12,000 rpm for 5 min. Protein from 5×10⁴ cells (25 μg) was subjected to electrophoresis on 7.5-10% SDS-polyacrylamide gels and electro-transferred to nitrocellulose membrane. Nonspecific binding was blocked by incubating with Tris-buffered saline (TBS) [137 mM NaCl, and 15 mM Tris-HCl (pH 7.6)] containing 5% non-fat milk (w/v) for 1 hr at room temperature and then probed overnight with primary antibodies against COX-1, COX-2, and phosphor-p65 NF-κB. After washing in TBS-Tween 20 buffer (0.075% v/v), the membranes were incubated with horseradish peroxidase-conjugated anti-IgG antibody. Proteins bound to the primary antibodies were detected by the enhanced chemiluminescence (ECL) detection system and quantified by densitometric scanning.

Measurement of PGE₂ Production: The PGE2 levels in cell culture medium collected as described above were measured using a competitive enzyme immunoassay (EIA) following the manufacturer's instruction.

(ii) Effects of Phenolic Compounds and Methyl Ester of Avenanthramides on Colon Cancer Cells

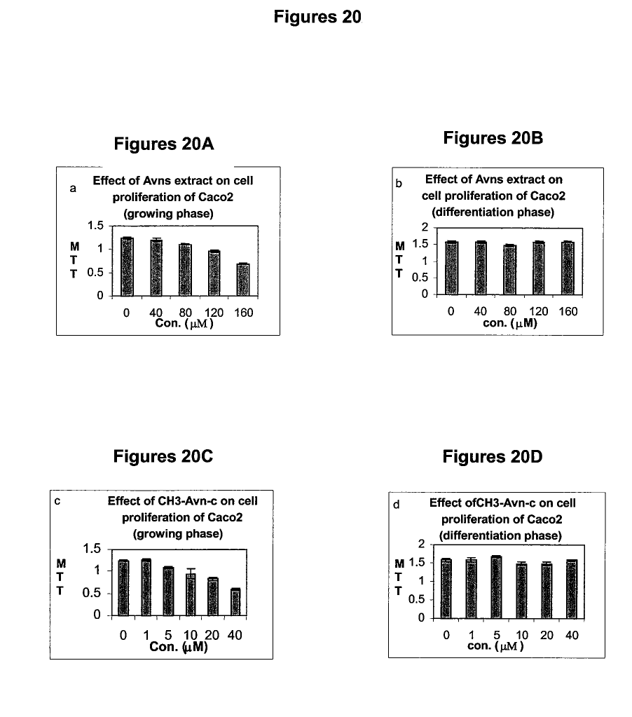

Effect of Avns on cell proliferation. The growth effect of Avns oat extract and CH₃-Avn-C was detected using human colon cancer cell lines CaCo2, HCT116, and HT-29. One important property of CaCoo-2 cells is that they can become differentiated by incubating confluent cells for 7-10 days, which is a good model to study the effect of chemotherapeutic agents on cancer versus its normal phenotype cells (Rousset, M., The human colon carcinoma cell lines HT-29 and Caco-2: two in vitro models for the study of intestinal differentiation. Biochimie, 1986. 68(9): p. 1035-40). Growing phase Caco2 cells (FIGS. 20a and 20c) and confluence-induced differentiated Caco2 (FIGS. 20b and 20d) cells were treated with different concentrations of either Avns extract (FIGS. 20a and 20b) or CH₃-Avn-C (FIG. 1c and 1d) in the 10% FCS medium for 48 h. MTT assay were performed to determine the cell proliferation. Data were means ±SEM from three separate experiments.

As shown in FIG. 20C, after 48 h of treatment, CH₃-Avn-C dose-dependently inhibited proliferation of growing phase of CaCo-2 cell, which displays cancer cell characteristics. At 40 μM, it inhibited more than 50% of cell growth. Interestingly, these compounds had no effect on the differentiated cells, which represents the normal epithelial colonic cells (FIG. 20D). Similar results were also obtained from Avns oat extract (FIGS. 20A and 20B) but at a higher concentration (IC₅₀ of 120-160 μM).

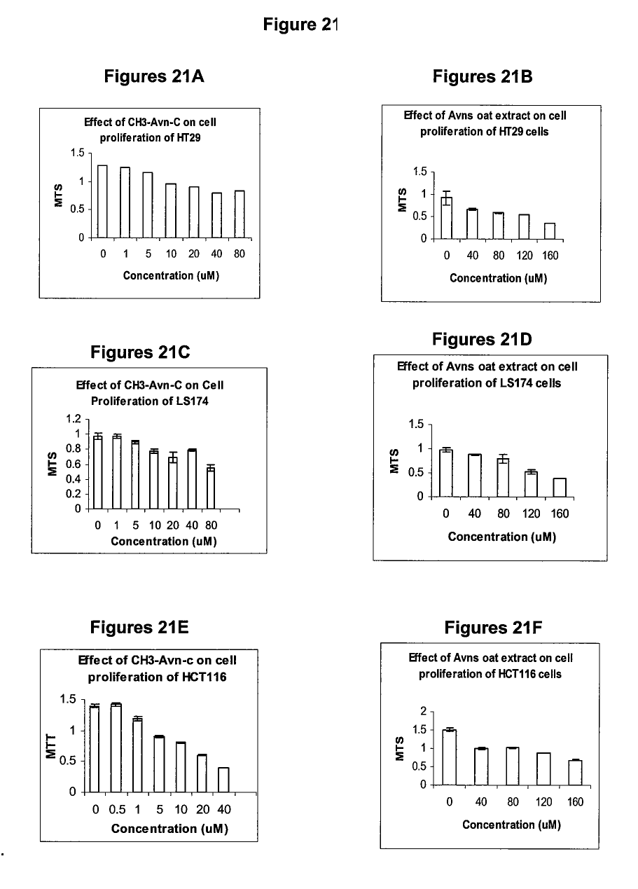

The same number of growing phase colon cancer cell lines, HT29 (FIGS. 21A&B), LS174 (FIGS. 21C&D), and HCT116 (FIGS. 21E&F) seeded into 96-well plate were treated with different concentrations of CH₃-Avn-C (FIGS. 21A, C, & E) or Avn oat extract (FIGS. 21B, D, & F) in the 10% FCS medium for 48 h. MTT assay were performed to determine the cell proliferation. Data were means ±SEM from three separate experiments.

Both CH₃-Avn-C (FIGS. 21A, C, & E) and Avns oat extract (FIGS. 21B, D, & F) significantly and dose dependently inhibited cell proliferation of HCT116, LS-174, and HT29 colon cancer cells. The IC₅₀ of CH₃-Avn-C for HCT116, LS-174, and HT29 is 10, 80 and >80 μM, respectively. HCT116 is COX-2 negative and both LS-174 and HT29 are COX-2 positive. Some polyphenols inhibited COX-2 positive colon cancer cell growth but have no effect on COX-2 negative colon cancer cells. Our results suggest that the inhibitory effect of Avns on colon cancer cell proliferation is independent of COX-2 pathway.

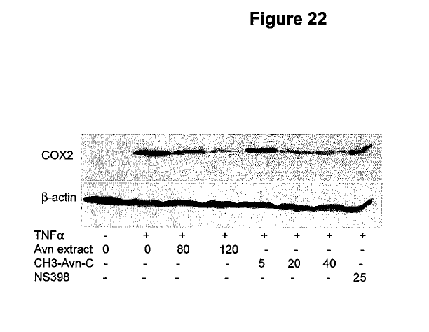

Effect of Avns on COX-2 expression: HT29 cells express a low basal level of COX-2 protein. TNF-α (40 μg/mL) stimulation upregulated its expression. Seventy percent confluent cells were pre-treated with different concentrations of Avns oat extract and CH₃-Avn-C for 1 hr followed by stimulation with 40 ng/ml of TNF-α for 23 hr. Whole cell lysates were prepared as described in method section. The expression of COX2 was measured by Western blotting. β-Actin expression level was used as a loading control. As shown in FIG. 22, both CH₃-Avn-C and Avns oat extract inhibited TNF-α induced COX-2 expression. This inhibitory effect is dose-dependent. At 10 μM concentration, CH₃-Avn-C inhibited 70% of COX2 protein expression. NS398, a specific COX2 inhibitor, only inhibited 50% of COX2 expression in our experiments at the concentration of 25 μM, a concentration commonly used. Avns oat extract have a similar inhibitory effect on COX2 expression, but at a higher concentration (120 μM). This inhibitory effect of Avns on COX2 expression is not due to the toxicity because at 24 hr treatment, the cell viability did not change at each concentration indicated.

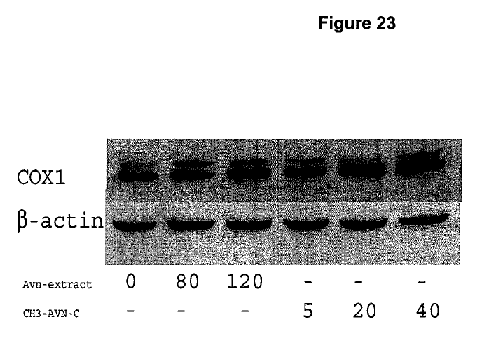

Effect of Avns on COX1 protein expression: COX1 and COX2 play different roles in the pathophysiology of the intestine. While COX2 is induced at the site of inflammation and contribute to its initiation and progression, COX1 is constitutively expressed and contributes to the production of intestine mucosa. The side effect of NSAIDs to the intestines is through the inhibition of COX1 and disruption of the mucous-bicarbonate barrier and mucosal integrity (Gudis, K. and C. Sakamoto, The role of cyclooxygenase in gastric mucosalprotection. Dig Dis Sci, 2005. 50 Suppl 1: p. S16-23). Some polyphenols, such as curcumin and EGCG, have been shown to inhibit COX2 expression but have no effect on COX1 (Lev-Ari, S., et al., Celecoxib and curcumin synergistically inhibit the growth of colorectal cancer cells. Clin Cancer Res, 2005. 11(18): p. 6738-44). As shown in FIG. 23, the basal level of COX1 expression in HT29 cells (without TNF-α stimulation) is abundant, treatment with different concentrations of Avns have no significant effects on COX1 protein expression level. This data provided a reasonable assumption that Avns has a high capacity being as a specific COX2 inhibitor which might be devoid for the side effects associated with NSAIDs. Seventy percent confluent HT29 colon cancer cells were treated with different concentrations of Avns oat extract and CH₃-Avn-C for 24 hr. Whole cell lysates were prepared as described in method section. The expression of COX1 was measured by Western blotting. β-Actin expression level was used as a loading control.

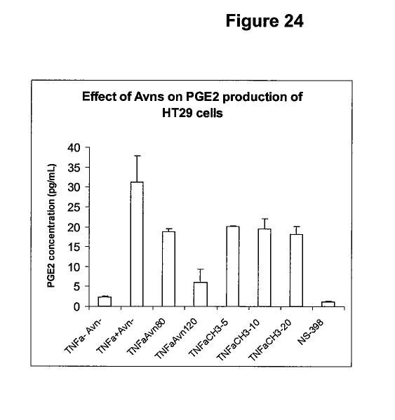

Effect of Avns on PGE2 production. It has been reported that COX2 metabolite PGE2 played an important role in colorectal tumorigenesis and promoting colorectal tumor cell growth (Sheng, H., et al., Prostaglandin E2 increases growth and motility of colorectal carcinoma cells. J Biol Chem, 2001. 276(21): p. 18075-81; Wang, D., et al., Prostaglandin E2 enhances intestinal adenoma growth via activation of the Ras-mitogen-activated protein kinase cascade. Cancer Res, 2005. 65(5): p. 1822-9). Since we found that COX2 was down-regulated by Avns, we next examined whether Avns have an effect on PGE2 production. The effect of Avns on PGE2 production levels in the cell culture medium collected from HT29 cells used for COX2 Western blot were determined using a PGE2 EIA kit. As shown in FIG. 24, there was more than 10-fold increase in PGE2 level in TNF-α treated cells. This increase in PGE2 by TNF-α stimulation was inhibited by pre-treatment with either Avns extract or CH₃-Avn-C. At 120 μM of Avns oat extract, around 80% of PGE2 in HT29 cell culture medium was inhibited.

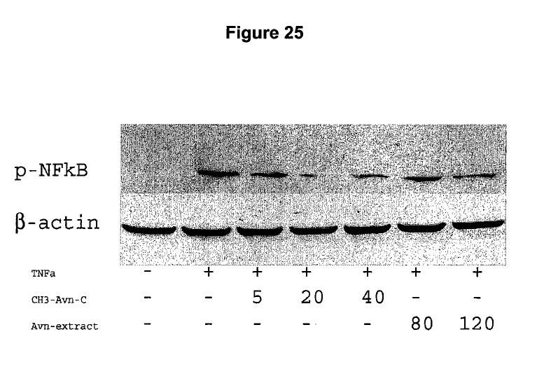

Effect of Avns on NFκB activity: The effect of Avns treatment on NFκB activity was explored through detecting p65 subunit phosphorylation using western blot. Seventy percent confluent cells were pre-treated with different concentrations of Avns oat extract and CH₃-Avn-C for 23 hr followed by stimulation with 40 ng/ml of TNF-α for 40 min. Whole cell lysates were prepared as described in method section. The NFκB phosphorylation was measured by Western blotting. β-Actin expression level was used as a loading control. As shown in FIG. 25, pretreatment with Avns inhibited around 60% of the TNF-α induced phosphorylation of p65 subunit of NFκB compared to that of untreated cells. Similarly, the COX2 expression induced by TNF-α in HT29 cells has been found to be NFkB dependent (Jobin, C., et al., Specific NF-kappaB blockade selectively inhibits tumour necrosis factor-alpha-induced COX-2 but not constitutive COX-1 gene expression in HT-29 cells. Immunology, 1998. 95(4): p. 537-43).

In summary, this Example shows that both Avns oat extract and synthesized CH₃-Avn-C inhibit colon cancer cell proliferation in a dose-dependently manner. Interestingly, they had no significant inhibitory effect on confluence-induced well differentiated colon cancer cell which represents normal colon epithelial cells. Both basal level and TNF-α induced COX2 was significantly inhibited by Avns oat extract or CH₃-Avn-C. This growth inhibitory effect may at least partially be due to its inhibiting of NF-κB activity. The results show that anti-inflammatory properties of Avns may at least partially contribute to the health benefits of oats in reducing colon cancer risk. Furthermore, this study shows that compositions comprising phenolic compounds, such as CH₃-Avn-C, can be used as a naturally-based anti-cancer drug for colon cancer.

While the present invention has been described in terms of specific methods and compositions, it is understood that variations and modifications will occur to those skilled in the art upon consideration of the present invention. Those skilled in the art will appreciate, or be able to ascertain using no more than routine experimentation, further features and advantages of the invention based on the above-described embodiments. Accordingly, the invention is not to be limited by what has been particularly shown and described. All publications and references are herein expressly incorporated by reference in their entirety.

1. A method for decreasing proliferation of human colon adenocarcinoma cells in a subject comprising
administering to a subject an effective amount of a substantially purified composition of at least one member selected from the group consisting of compounds of formula: 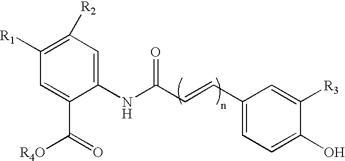
wherein n is less than or equal to six,
R₁, R₂, and R₃ are the same or different and selected from the group consisting of a hydrogen, a hydroxide, an aliphatic group, an aromatic group, an acyl group, an alkoxy group, an alkylene group, an alkenylene group, an alkynylene group, a hydroxycarbonylalkyl group, an anhydride, an amide, an amine, and a heterocyclic aromatic group, and
R₄ is a hydrogen or an alkyl group.

2. The method of claim 1, wherein n is less than 3 and R₁, R₂, and R₃ can be selected from the group consisting of H, OH, and OCH₃.

3. The method of claim 1, wherein the method further comprises administering at least one compound that is an extract or concentrate of oat grain.

4. The method of claim 1, wherein the method further comprises administering at least one compound that is produced synthetically.

5. The method of claim 1, wherein n is 1, R₁ is OH, R₂ is hydrogen, R₃ is OH, and R₄ is a lower alkyl group.

6. The method of claim 5, R₄ is a methyl group.

7. The method of claim 1, wherein n is 1, R₁ is OH, R₂ is hydrogen, R₃ is OH, and R₄ is hydrogen.

8. The method of claim 1, wherein administration is selected from the group consisting of oral administration, transdermal administration to said adenocarcinoma cells, and injection into said adenocarcinoma cells.

9. The method of claim 1, wherein the administration selectively inhibits production of COX-2.

10. The method of claim 9, wherein the administration inhibits production of prostaglandin E2 (PGE2) levels.

11. The method of claim 1, wherein the method further comprises inhibiting NFκB phosphorylation.

12. A method for reducing risk and delaying onset of colon cancer in a subject, comprising:
administering to a subject an effective amount of a substantially purified phenolic composition of formula, 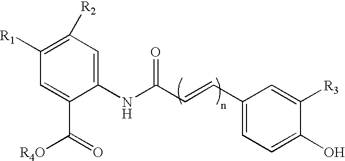
wherein n is less than or equal to six,
R₁, R₂, and R₃ are the same or different and selected from the group consisting of a hydrogen, a hydroxide, an aliphatic group, an aromatic group, an acyl group, an alkoxy group, an alkylene group, an alkenylene group, an alkynylene group, a hydroxycarbonylalkyl group, an anhydride, an amide, an amine, and a heterocyclic aromatic group, and
R₄ is a hydrogen or an alkyl group,
whereby administration inhibits NFκB phosphorylation in colon adenocarcinoma cells.

13. The method of claim 12, wherein n is less than three and R₁, R₂, and R₃ are selected from the group consisting of H, OH, and OCH₃.

14. The method of claim 12, wherein the method further comprises administering at least one compound that is an extract or concentrate of oat grain.

15. The method of claim 12, wherein the method further comprises administering at least one compound that is produced synthetically.

16. The method of claim 12, wherein n is 1, R₁ is OH, R₂ is hydrogen, R₃ is OH, and R₄ is a lower alkyl group.

17. The method of claim 16, wherein R₄ is a methyl group.

18. The method of claim 12, wherein n is 1, R₁ is OH, R₂ is hydrogen, R₃ is OH, and R₄ is hydrogen.

19. The method of claim 12, wherein the subject is at risk to develop colon cancer.

20. The method of claim 12, wherein administration selectively inhibits production of COX-2.
